(19) United States
(12) Patent Application Publication
(10) Pub. No.: US 2023/0075521 A1
(43) Pub. Date: Mar. 9, 2023

(54) RESVERATROL PHARMACEUTICAL COMPOSITIONS AND METHODS OF USE THEREOF

(71) Applicant: Jupiter Neurosciences, Inc., Jupiter, FL (US)

(72) Inventors: Marshall A. Hayward, Bridgewater, NJ (US); Christopher J. Kemper, Lawrenceville, NJ (US)

(21) Appl. No.: 17/891,662

(22) Filed: Aug. 19, 2022

Related U.S. Application Data
(60) Provisional application No. 63/235,253, filed on Aug. 20, 2021.

(51) Int. Cl. A61K 31/05; A61K 9/00; A61K 47/26; A61K 47/14; A61K 47/22; A61P 25/14; A61P 3/00; A61P 25/28; A61P 25/00

(52) U.S. Cl. CPC A61K 31/05; A61K 9/0053; A61K 47/26; A61K 47/14; A61K 47/22; A61P 25/14; A61P 3/00; A61P 25/28; A61P 25/00

(57) ABSTRACT

Methods of enhancing the bioavailability of resveratrol, and for the treatment of at least one neuroinflammatory disorder in a subject, include orally administering to the subject a resveratrol solubulization product formulation consisting of: resveratrol; an emulsifying agent mixture of polysorbate 80 and polysorbate 20; at least one medium-chain triglyceride (MCT); and tocopherol or mixed tocopherols, or an oral pharmaceutical composition containing same. In the methods, the formulation is orally administered to the human subject under fasting conditions. Upon oral administration to a human subject under fasting conditions, the resveratrol solubulization product formulation or oral pharmaceutical composition containing same provides at least one of the following pharmacokinetic parameters: a. AUC₍₀₋ₜ₎ of at least about 500 h*ng/mL; b. AUC_{(0-infin.)} of no more than about 2100 h*ng/mL; and c. Cmax of at least about 220 ng/ml, wherein t is between about 1 and about 24 hours.

### CROSS-REFERENCE TO RELATED APPLICATIONS

This application claims the benefit of priority to U.S. Provisional Patent Application No. 63/235,253, filed Aug. 20, 2021, which is incorporated herein by reference in its entirety.

### STATEMENT REGARDING FEDERALLY SPONSORED RESEARCH

The invention described herein was supported in whole or in part by a grant from the National Institute on Aging of the United States National Institutes of Health, Grant Award 1R44AG067907-01A1. The U.S. Government has certain rights in this invention.

### FIELD OF THE INVENTION

The invention relates generally to the fields of pharmacology, medicine, and neurology. In particular, the invention relates to methods of enhancing the bioavailability of resveratrol in a subject and methods for the treatment of at least one neuroinflammatory disorder in a subject.

### BACKGROUND

Neuroinflammation is a causative issue in most neurological diseases such as Alzheimer's disease (AD) and in many rare diseases. Inflammation of the brain might not always be the root cause of the disease but is often a contributing factor of progression and severity in diseases such as, for example, AD, mild traumatic brain injury (TBI), and concussion. In AD, neuroinflammation increases with disease progression, causing cell and neurological damage. In mild TBI and concussion, neuroinflammation appears in most cases immediately after a blunt injury to the brain caused through falls, sports, and accidents. Many rare diseases are associated with neuroinflammation as well as other disease-specific problems. Only a small fraction of the more than 7000 known rare diseases have an effective treatment. For example, Friedreich's Ataxia is a CNS and mitochondrial disease with no currently approved treatment. Several other Ataxia indications are also lacking treatments. As another example, lysosomal storage disorders are treated with enzyme replacement therapies that effectively prolong life, but leave patients with cognitive disorders, rheumatoid arthritis, loss of eyesight and hearing and deteriorating quality of life. As yet another example, there are more than 10 mitochondrial rare diseases that are life threatening at various ages. Most, if not all, would benefit from a product that can boost mitochondrial function to prevent cell death.

### SUMMARY

Described herein are methods of enhancing the bioavailability of resveratrol in a subject, and methods for the treatment of at least one neuroinflammatory disorder in a subject. These methods include administration of a resveratrol solubulization product formulation containing about 200 mg to about 700 mg (e.g., about 500 mg) of resveratrol to a subject under fasting conditions. Previous use of resveratrol (nutritional resveratrol) has been limited by adverse side effects (e.g., gastrointestinal side effects) at doses required for positive physiological effects. As shown in the Examples below, improved bioavailability of resveratrol with the resveratrol solubulization product formulations described herein when administered to subjects under fasting conditions was observed compared to naturally occurring resveratrol. Also, in an AD mouse model, the experimental results showed that a resveratrol solubulization product formulation as described herein displays significantly increased bioavailability over non-formulated resveratrol. From the pharmacokinetics (PK) studies described in the Examples below, several unexpected findings were observed. First, while a dose proportional result was expected based on the conventional wisdom, absorption was not dose proportional. In fact, absorption very surprisingly increased disproportionally with higher doses. The finding that absorption is not dose proportional was surprising and is in fact beneficial, for as therapeutic doses are approached, proportionally lower administered doses (“GI tract burden”) are needed to achieve therapeutic blood levels. Second, use of the resveratrol solubulization product formulations described herein achieved therapeutically relevant blood levels. That is, blood (plasma) concentrations of resveratrol greater than 220 ng/ml at Cₘₐₓ—at levels of AUC (area under the curve) that are well below the Food and Drug Administration (FDA)-mandated limit of 2100 ng*hr/mL. At, for example, 500 mg resveratrol administered in a resveratrol solubulization product formulation as described herein, levels in the therapeutic range are expected based on the PK studies presented in the Examples below. These PK studies demonstrate that administration of resveratrol at a concentration of 200 mg to 700 mg delivered in a resveratrol solubulization product formulation as described herein to subjects under fasting conditions increased bioavailability of the resveratrol in plasma compared to nutritional resveratrol. Further, it was expected that there would be a positive food effect based on studies with nutritional resveratrol. But as demonstrated in the Examples below, bioavailability is surprisingly and negatively impacted by dosing with food. Therefore, the resveratrol solubulization product formulations described herein are administered to a subject in need thereof on a fasted/empty stomach. The resveratrol solubulization product formulation described herein are orally administered, safe and effective for use in many disease indications; when orally administered to a subject under fasting conditions, the resveratrol passes the Blood Brain Barrier where it exerts positive effects on oxidative stress, inflammation and mitochondrial function. By reducing the gastric tract burden, patient tolerability, compliance, and ultimately therapeutic outcome are improved.

Accordingly, described herein is a method of enhancing the bioavailability of resveratrol in a subject. The method includes orally administering to the subject (e.g., human subject) a resveratrol solubulization product formulation consisting of: resveratrol; an emulsifying agent mixture of polysorbate 80 and polysorbate 20; at least one medium-chain triglyceride (MCT); and tocopherol or mixed tocopherols. In the method, the formulation is orally administered to the subject under fasting conditions. In the method, the resveratrol administered can be in an amount from about 200 mg to about 700 mg per dose. The mg of resveratrol can be, e.g., 199, 200, 201, 225, 250, 275, 300, 325, 350, 375, 400, 425, 450, 475, 499, 500, 501, 502, 503, 504, 505, 525, 550, 575, 600, 625, 650, 675, 700, 701, 702. The mg of resveratrol can be within a range of any high value and any low value selected from these values. In some embodiments, the amount of resveratrol per dose is about 500 mg. In embodiments, the resveratrol solubulization product formulation is administered at least once (e.g., once, twice, three times) daily. The resveratrol solubulization product formulation can be formulated as, for example, a soft gelatin capsule, a hard gelatin capsule, a soft gelatin-free capsule, or a hard gelatin-free capsule. For example, to achieve a dose of about 500 mg resveratrol, 5 capsules can be administered.

In embodiments of the method, the resveratrol exhibits an AUC_{O-t} which is about 500 h*ng/ml to about 2000 h*ng/ml following administration of the resveratrol solubulization product formulation to the subject (e.g., human subject) under fasting conditions, wherein t is between about 1 and about 24 hours. In embodiments, the resveratrol exhibits a Cmax which is about 220 ng/ml to about 400 ng/ml (e.g., about 260 ng/ml to about 375 ng/ml, about 300 ng/ml to about 350 ng/ml) following administration of the resveratrol solubulization product formulation to the subject under fasting conditions. In embodiments, the resveratrol exhibits an AUC_{(0-infin.)} which is about 500 h*ng/mL to about 2100 h*ng/mL following administration of the resveratrol solubulization product formulation to the subject under fasting conditions. The fasting conditions can include fasting the subject for at least about 2 hours immediately prior to administering the formulation, and for at least another about 1 hour immediately after administering the formulation.

Also described herein is an oral pharmaceutical composition. The oral pharmaceutical composition includes at least one dose of a resveratrol solubulization product formulation consisting of: about 200 to about 700 mg of resveratrol; an emulsifying agent mixture of polysorbate 80 and polysorbate 20; at least one MCT; and tocopherol or mixed tocopherols, wherein the formulation is formulated as a soft gelatin capsule, a hard gelatin capsule, a soft gelatin-free capsule, or a hard gelatin-free capsule. The oral pharmaceutical composition, upon oral administration to a human subject under fasting conditions, provides at least one of the following pharmacokinetic parameters: a. AUC₍₀₋ₜ₎ of at least about 500 h*ng/mL; b. AUC_{(0-infin.)} of no more than 2100 h*ng/mL; and c. Cmax of at least about 220 ng/ml (e.g., about 220 ng/ml to about 400 ng/ml, about 260 ng/ml to about 375 ng/ml, about 300 ng/ml to about 350 ng/ml), wherein t is between about 1 and 24 hours. An oral pharmaceutical composition as described herein can include multiple doses (e.g., 2, 3, 4, 5, 10, 15, 20, 50, 100 doses etc.) of the resveratrol solubulization product formulation, each dose containing about 200 to about 700 mg of resveratrol; an emulsifying agent mixture of polysorbate 80 and polysorbate 20; at least one MCT; and tocopherol or mixed tocopherols.

Further described herein is a method for the treatment of at least one neuroinflammatory disorder. The method includes orally administering to a human subject in need thereof an oral pharmaceutical composition as described herein. In the method, the formulation is orally administered to the human subject under fasting conditions. In embodiments, the at least one neuroinflammatory disorder is one or more of: Ataxia, Alzheimer's disease, Mild Cognitrive Impairment, ALS, Parkinsonism, an acute neurologic injury, TBI including concussion, a Lysosomal Storage Disease such as mucopolysaccharidosis type I, III, IV, or VII, a mitochondrial function disorder, MELAS, LHON, hearing loss, and speech acuity.

By the term “resveratrol solubulization product formulation as described herein” is meant resveratrol formulation including about 200 to about 700 mg of resveratrol; an emulsifying agent mixture of polysorbate 80 and polysorbate 20; at least one MCT; and tocopherol or mixed tocopherols, wherein upon oral administration to a human subject under fasting conditions, provides at least one of the following pharmacokinetic parameters: a. AUC₍₀₋ₜ₎ of at least about 500 h*ng/mL; b. AUC_{(0-infin.)} of no more than 2100 h*ng/mL; and c. Cmax of at least about 220 ng/ml (e.g., about 220 ng/ml to about 400 ng/ml, about 260 ng/ml to about 375 ng/ml, about 300 ng/ml to about 350 ng/ml), wherein t is between about 1 and about 24 hours. A resveratrol solubulization product formulation as described herein can be formulated as, e.g., a soft gelatin capsule, a hard gelatin capsule, a soft gelatin-free capsule, or a hard gelatin-free capsule. In the Examples below, the resveratrol solubulization product formulations as described herein are referred to as JOTROL™ (Jupiter Neurosciences, Jupiter, Fla., USA) and are commercially available from Jupiter Neurosciences (Jupiter, Fla., USA).

As used herein, the term “medium-chain triglyceride (MCT)” means any triglyceride containing medium-chain fatty acids. Medium-chain fatty acids include capronic acid, caprylic acid, capric acid, and lauric acid. These are saturated fatty acids, which are present in tropical plant fats such as coconut oil and palm kernel oil. Low fractions of the substances are also present in milk fat. There is no pure MCT oil in nature, but pure MCT oils can be obtained by synthesis. In the resveratrol solubulization product formulation as described herein, individual MCTs or a mixture of different MCTs can be used as medium-chain triglycerides.

As used herein, the terms “resveratrol available in the nutritional market”, “nutritional resveratrol”, and “regular resveratrol” mean any resveratrol available in the market in a formulation that is not manipulated or formulated to enhance bioavailability. This includes micronized formulations.

As used herein, the terms “neuroinflammatory”, “neuro inflammation” and “neurologic inflammation” are used interchangeably and mean inflammation of the brain, spinal cord, central nervous system, or inflammation associated with any neurologic condition.

By the term “therapeutically relevant blood level” is meant a blood concentration of resveratrol greater than 220 ng/ml (e.g., about 220 ng/ml to about 400 ng/ml, about 260 ng/ml to about 375 ng/ml, about 300 ng/ml to about 350 ng/ml) at Cₘₐₓ at a level of AUC below 2100 ng*hr/mL.

As used herein, the term “fasting conditions” means an empty stomach. A stomach containing water is considered “empty”. Generally, the fasting conditions include fasting the subject for at least 2 hours immediately prior to administering the formulation, and at least about 1 hour after administration.

As used herein, the term “bioavailability” refers to the extent and rate at which the active moiety (e.g., resveratrol) enters systemic circulation, thereby accessing the site of action.

The terms “patient,” “subject” and “individual” are used interchangeably herein, and mean a subject, typically a mammal, to be treated, diagnosed, and/or to obtain a biological sample from. Subjects include, but are not limited to, humans, non-human primates, horses, cows, sheep, pigs, rats, mice, insects, dogs, and cats. A human in need of neurologic inflammation treatment is an example of a subject (e.g., a human suffering from AD, mild TBI, concussion, Friedreich's Ataxia, etc.).

The terms “sample,” “patient sample,” “biological sample,” and the like, encompass a variety of sample types obtained from a patient, individual, or subject and can be used in a diagnostic or monitoring assay. The patient sample may be obtained from a healthy subject, a diseased patient or a patient having associated symptoms of a particular disease or disorder (e.g., a neurological disorder such as, e.g., AD, mild TBI, concussion, Friedreich's Ataxia, etc.). The definition specifically encompasses blood and other liquid samples of biological origin (including, e.g., cerebrospinal fluid, plasma, serum, peripheral blood), solid tissue samples such as a biopsy specimen or tissue cultures or cells derived therefrom and the progeny thereof. In an embodiment, a sample includes a cerebrospinal fluid sample.

As used herein, the terms “therapeutic treatment” and “therapy” are defined as the application or administration of a therapeutic agent (e.g., a resveratrol solubulization product formulation as described herein) or therapeutic agents (e.g., a resveratrol solubulization product formulation as described herein and another therapeutic) to a patient who has a disease, a symptom of disease or a predisposition toward a disease, with the purpose to cure, heal, alleviate, relieve, alter, remedy, ameliorate, improve or affect the disease, the symptoms of disease, or the predisposition toward disease. For example, administration of a therapeutic agent (a resveratrol solubulization product formulation as described herein) to a subject susceptible to AD or in the early clinical stages of AD may delay the onset or progression of AD. A “therapeutically effective amount” of a resveratrol solubulization product formulation as described herein is the amount necessary to cure, heal, alleviate, relieve, alter, remedy, ameliorate, improve or affect a disease, the symptoms of disease, or the predisposition toward disease, and is typically in the range of about 200 to about 700 mg of resveratrol per dose administered at least once (e.g. once, twice, three times, etc.) daily.

The term “about” in reference to a numerical value refers to the range of values somewhat less or greater than the stated value, as understood by one of skill in the art. For example, the term “about” could mean a value ranging from plus or minus a percentage (e.g., .+−0.1%, 2%, or 5%) of the stated value. Unless otherwise indicated, all presented values may be understood as modified by the term “about.”

Although methods, formulations, and compositions similar or equivalent to those described herein can be used in the practice or testing of the present invention, suitable methods, formulations, and compositions are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a graph showing Phase 1 PK study results of administration of a resveratrol solubulization product formulation as described herein under fasting conditions compared to nutritional resveratrol (MCRI) administered at 2.5 grams resveratrol twice daily in a Friedreich's Ataxia study (‘MCRI 5g’—Yiu et al., J Neurol. 2015; 262(5):1344-53), and MCRI administered at 1.0 gram resveratrol twice daily in an AD study (‘Turner 2g’—Turner et al., Neurology 2015; 85: 1383-1391). 250 ng/ml in blood plasma at C-Max is seen as a minimum to achieve good therapeutic effect.

FIG. 2 is a series of graphs showing log-linear concentration (ng/mL) vs. time plots of Resveratrol (RES) and its metabolites Resveratrol Sulfate (3S_RES), Resveratrol 3-Glucuronide (3G_RES) and Resveratrol 4-Glucuronide (4G_RES) in Human Plasma.

FIG. 3 is a series of column plots of Cmax (ng/mL), AUC (ng·hr/mL), amount (Ae0-t (ng)) and fraction collected in urine of RES, 3_RES, 3S_RES, and 4G_RES.

FIG. 4A-4D is a graph and a series of plots showing short-term treatment of ged (14 months) male 3×Tg-AD mice increases Adam10 expression and decreases inflammation in liver. The graph represents mean+SEM. *p<0.05, n=3-5.

FIG. 5 is a series of graphs showing sensorimotor and anxiety-related behavior is unchanged between JOTROL and vehicle mice while differences in object location memory are seen between JOTROL and RSV mice. The graphs represent mean+SEM. *p<0.05; n-8-10/group.

FIGS. 6A and 6B are a series of graphs showing AD pathological hallmarks by sex and brain region. Aβ42 levels are significantly decreased in the ERC of JOTROL-treated females only, and unchanged between treatment groups in PFC and hippocampus. Total tau is significantly decreased in the hippocampus of RSV-treated males. Phospho-tau at Ser396 is significantly different in PFC between JOTROL- and RSV-treated females and between vehicle and RSV-treated males. The graphs represent mean±SEM. Males and females of the same treatment group were analyzed using two-way unpaired t-test or Mann-Whitney test where appropriate (*p<0.05, ##p<0.01 for significance between sex within treatment group).

FIG. 7A-7E is a plot and a series of graphs showing Long-term JOTROL treatment improves AD-related gene expression. FIG. 7A: Sirt1 mRNA expression is significantly increased in JOTROL and RSV groups compared to vehicle. FIG. 7B: Sirt1 activity shows a trend towards increased activity in the brains of JOTROL mice. FIG. 7C-7E: mRNA expression in PFC, Hippocampus, and ERC. The graphs represent mean±SEM. *p<0.05, **p<0.01, ***p<0.001, ****p<0.0001; ns, not significant, n=9-10/group.

FIG. 8 is a series of graphs showing changes in cytokine and chemokine levels in serum and ERC as detected by Luminex immune panel. The graphs represent mean±SEM. *p<0.05, **p<0.01; n=8-10/group.

### DETAILED DESCRIPTION

Described herein are resveratrol solubulization product formulations and oral pharmaceutical compositions, and methods of administering them to a subject in need thereof under fasting conditions to enhance the bioavailability of resveratrol, and to treat at least one neuroinflammatory disorder (e.g., AD). Resveratrol is a phytoalexin with anti-inflammatory properties and is a polyphenol. In the Examples below, administration of the resveratrol solubulization product formulations described herein under fasting conditions resulted in improved bioavailability of resveratrol, absorption that was not dose proportional, therapeutically relevant blood levels, and bioavailability that was negatively impacted by dosing with food. Additionally, in an AD mouse model, a resveratrol solubulization product formulation as described herein displayed significantly increased bioavailability over non-formulated resveratrol. It was observed that both sub-chronic and long-term treatment with JOTROL™ improved several AD-related genes and impacts central and systemic inflammation in the AD mouse model.

### Oral Pharmaceutical Compositions

Oral pharmaceutical compositions containing the resveratrol solubulization product formulations are described herein. The resveratrol solubulization product formulations described herein include micelles that are loaded with resveratrol due to the polysorbate 80, polysorbate 20, at least one MCT, and tocopherol or mixed tocopherols. Conventional methods of administering nutritional or native resveratrol include administration of such a high dose (e.g., 1 gram, 1.5 gram, 2 grams, greater than 2 grams, etc.) that adverse side effects (e.g., abdominal pain, diarrhea, etc.) result. The resveratrol solubulization product formulations described herein when administered under fasting conditions provide improved bioavailability of resveratrol, proportionally lower administered doses and thus a lower GI tract burden, while achieving therapeutic blood levels. Typically, therapeutic blood levels are blood concentrations of resveratrol greater than 220 ng/ml (e.g., about 220 ng/ml to about 400 ng/ml, about 260 ng/ml to about 375 ng/ml, about 300 ng/ml to about 350 ng/ml) at Cₘₐₓ having a level of AUC that is below the limit of 2100 ng*hr/mL.

A typical oral pharmaceutical composition as described herein includes at least one dose of a resveratrol solubulization product formulation consisting of: about 200 to about 700 mg of resveratrol; an emulsifying agent mixture of polysorbate 80 and polysorbate 20; at least one MCT; and tocopherol or mixed tocopherols. In a typical oral pharmaceutical composition, the resveratrol solubulization product formulation is formulated as a soft gelatin capsule, a hard gelatin capsule, a soft gelatin-free capsule, or a hard gelatin-free capsule. The composition, upon oral administration to a human subject under fasting conditions, provides at least one of the following pharmacokinetic parameters: a. AUC₍₀₋ₜ₎ of at least about 500 h*ng/mL; b. AUC_{(0-infin.)} of no more than about 2100 h*ng/mL; and c. Cₘₐₓ of at least about 220 ng/ml (e.g., about 220 ng/ml to about 400 ng/ml, about 260 ng/ml to about 375 ng/ml, about 300 ng/ml to about 350 ng/ml), wherein t is between about 1 and about 24 hours.

An oral pharmaceutical composition as described herein can include multiple doses (e.g., 2, 3, 4, 5, 6, 7, 8, 9, 10 doses, etc.) of the resveratrol solubulization product formulation, each dose containing about 200 to about 700 mg (e.g., about 500 mg) of resveratrol; an emulsifying agent mixture of polysorbate 80 and polysorbate 20; at least one MCT; and tocopherol or mixed tocopherols. In some embodiments, the amount of resveratrol per dose is about 500 mg. Any suitable source of resveratrol can be used. For example, commercially available (trans-) resveratrol, 99%, can be used. The resveratrol content in a typical resveratrol solubulization product formulation as describe herein is in the range of 3% by weight to 15% by weight of the resveratrol solubulization product formulation. The weight % of resveratrol can be, e.g., 2.9, 3.0, 3.1, 3.2, 3.3, 3.4, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, or 15.1%. The weight % of the resveratrol can be within a range of any high value and any low value selected from these values. In some embodiments, the resveratrol content is in the range of 5% by weight to 10% by weight, e.g., 10% by weight.

Any suitable source of polysorbate 80 and polysorbate 20 can be used, e.g., TEGO SMO 80 V, Evonik or Crillet 4/Tween 80-LQ-(SG); and TEGO SML 20 V, Evonik or Crillet 1/Tween 20-LQ-(SG). The emulsifying agent mixture of polysorbate 80 and polysorbate 20 can be in the range of approximately 65% by weight to approximately 95% by weight of the resveratrol solubulization product formulation as described herein. The weight % of the mixture of polysorbate 80 and polysorbate 20 can be e.g., 64.5, 64.9, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95%. The weight % of the mixture of polysorbate 80 and polysorbate 20 can be within a range of any high value and any low value selected from these values. For example, the % weight of the mixture of polysorbate 80 and polysorbate 20 can be in the range of approximately 70% by weight to approximately 92% by weight, e.g., the fraction of the emulsifying agent mixture can be approximately 71.8% by weight of the resveratrol solubulization product formulation as described herein.

For the at least one MCT, any suitable MCT can be used, e.g., MCT oil (70/30) Rofetan GTCC 70/30. The amount of the at least one MCT can be in the range of at least approximately 2% by weight to approximately 8% by weight. The weight % of the at least one MCT can be e.g., 1.9, 2.0, 2.1, 2.2, 2.3, 2.4, 2.5, 3.0, 3.5, 4.0, 4.5, 5.0, 5.5, 6.0, 6.5, 7.0, 7.5, 8.0, or 8.1%. The weight % of the at least one MCT can be within a range of any high value and any low value selected from these values. For example, the amount of the at least one MCT can be in the range of approximately 3% by weight to approximately 5% by weight, e.g., the fraction of the at least one MCT can be approximately 4.5% by weight.

Any suitable source of tocopherol can be used, e.g., Vitapherole T-70 Non GMO, a 70% mixed tocopherols in plant oil. The amount of tocopherol, in particular mixed tocopherols, is typically in the range of up to approximately 10% by weight of the resveratrol solubulization product formulation. The weight % of tocopherol or a mixture of tocopherols can be e.g., 0.01, 0.1, 0.25, 0.51, 1.0, 2.0, 3.0, 4.0, 5.0, 6.0, 7.0, 8.0, 9.0, 10.0, or 10.1%. The weight % of tocopherol or a mixture of tocopherols can be within a range of any high value and any low value selected from these values. In some embodiments, the amount of the tocopherol content is in the range of approximately 3% by weight to approximately 6% by weight, e.g., the tocopherol fraction can be approximately 5.25% by weight.

Micellar resveratrol formulations consisting of: resveratrol; an emulsifying agent mixture of polysorbate 80 and polysorbate 20; at least one MCT; and tocopherol or mixed tocopherols for use as a pharmaceutical product are described in U.S. Pat. No. 10,780,056, which is incorporated by reference herein in its entirety. Methods for preparing the micellar resveratrol formulations are also described in U.S. Pat. No. 10,780,056, and can be used to prepare the resveratrol solubulization product formulations described herein. In one embodiment of these methods, a micellar resveratrol formulation is prepared as follows:
100 g resveratrol; 45 g medium-chain triglycerides; 600 g polysorbate 80; 180 g polysorbate 20, and 75 g mixed tocopherols are used.
The resveratrol is (trans-)resveratrol, 99%, CAS number 501-36-0, procured from Evolva, Reinach Switzerland.
MCT oil (70/30) Rofetan GTCC 70/30 made by DHW Deutsche Hydrierwerke Rodleben GmbH, Dessau-RoJlau, Germany, CAS number 73-398-61-5, is used as the medium-chain triglycerides.
Commercial preparations such as, for example, TEGO SMO 80 V, Evonik or Crillet 4/Tween 80-LQ-(SG), Croda GmbH, Nettetal, Germany, can be used as polysorbate 80 (E433, CAS number 9005-65-6).
Commercial preparations such as, for example, TEGO SML 20 V, Evonik or Crillet 1/Tween 20-LQ-(SG), Croda GmbH, Nettetal, Germany, can be used as polysorbate 20 (E432, CAS number 9005-64-5). Vitapherole T-70 Non GMO, a 70% mixed tocopherols in plant oil made by Vitae Caps S.A., Spain, or EMix 70 made by Nutrilo GmbH, Cuxhaven, Germany, can be used as mixed tocopherols (E306, CAS numbers 59-02-9, 16698-35-4, 54-28-4, and 119-13-1).
Polysorbate 20, polysorbate 80, mixed tocopherols, and MCT oil are homogenized at a temperature in the range of approximately 18° C. to approximately 22° C. while stirring.
Resveratrol is then added to the mixture of polysorbate 20, polysorbate 80, mixed tocopherols, and MCT oil and heated, while stirring, to a temperature in the range of approximately 83° C. to approximately 87° C. for homogenization. As soon as the fluid is homogeneous and transparent, it is cooled to a temperature below approximately 30° C.
The resulting solubilization product is a light brown viscous fluid, which produces a yellowish clear solution when diluted with water at a ratio of 1:50. According to an HPLC analysis, the resveratrol content of the solubilization product is at least 10% by weight, whereby the resveratrol is enclosed in micelles. According to an aerometer measurement, the density of the solubilization product is in the range of 1.05 to 1.15 g/cm³ at a temperature of 20° C. The turbidity of the solubilization product is less than or equal to 50 FNU, solution in water at a ratio of 1:50. The solution has a pH in the range of 6 to 8 according to a potentiometric determination.

### Methods of Enhancing the Bioavailability of Resveratrol and Treating at Least One Neuroinflammatory Disorder in a Subject

Methods of enhancing the bioavailability of resveratrol in a subject, and treating at least one neuroinflammatory disorder in a subject include orally administering to the subject, under fasting conditions, a resveratrol solubulization product formulation as described herein, or an oral pharmaceutical composition containing a resveratrol solubulization product formulation as described herein. In the methods, the amount of resveratrol administered is typically in an amount from about 200 mg to about 700 mg per dose. In some embodiments, the resveratrol is in an amount of about 500 mg per dose. In the methods, the resveratrol solubulization product formulation as described herein, or an oral pharmaceutical composition containing same, is administered to the subject at least once daily. Depending upon a number of factors, in some embodiments specific to the subject (e.g., disorder, severity of symptoms, comorbidities), a resveratrol solubulization product formulation as described herein, or an oral pharmaceutical composition containing same, can be administered as many times a day (e.g., twice a day, three times a day, etc.) as necessary to treat the neuroinflammatory disorder. In the methods, after administration of a resveratrol solubulization product formulation as described herein, or an oral pharmaceutical composition containing same, the resveratrol exhibits an AUC_{O-t} which is about 500 h*ng/ml to no more than about 2100 h*ng/ml following administration of the formulation to the human subject under fasting conditions, wherein t is between about 1 and about 24 hours. The resveratrol also exhibits a Cmax which is about 220 ng/ml to about 400 ng/ml (e.g., about 220 ng/ml to about 400 ng/ml, about 260 ng/ml to about 375 ng/ml, about 300 ng/ml to about 350 ng/ml), and an AUC_{(0-infin.)} which is about 500 h*ng/mL to about 2000 h*ng/mL following administration of the formulation to the human subject under fasting conditions.

Generally, the fasting conditions include fasting the subject for at least 2 hours immediately prior to administering the formulation. The at least 2 hours immediately prior to administering the formulation can be e.g., 1.9, 2.0, 2.1, 2.2, 2.3, 2.4, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.1 hours, etc. In some embodiments, the at least two hours immediately prior to administering the formulation are an overnight fast. The fasting conditions also include fasting the subject for at least about 1 hour immediately after administering the formulation. The about 1 hour immediately after administration can be e.g., 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.1 hours, etc. The amount of time (hours, fraction of hours) fasting prior to and after administration can be within a range of any high value and any low value selected from the above values.

Typically in the methods, the subject is a human suffering from a neuroinflammatory disorder. A non-limiting list of neuroinflammatory disorders treatable with the resveratrol solubulization product formulations and oral pharmaceutical compositions described herein includes Ataxia, AD, Mild Cognitive Impairment, ALS, Parkinsonism, an acute neurologic injury, TBI including concussion, a Lysosomal Storage Disease such as mucopolysaccharidosis type I, III, IV, or VII, a mitochondrial function disorder, MELAS, LHON, hearing loss, speech acuity, etc., including combinations thereof.

Any suitable methods of administering the resveratrol solubulization product formulations and oral pharmaceutical compositions described herein to a subject may be used. In these methods, the resveratrol solubulization product formulations and compositions described herein are administered orally. In the Examples described below, it was discovered that bioavailability of resveratrol is negatively impacted by dosing with food. Therefore, the resveratrol solubulization product formulations described herein and oral pharmaceutical compositions containing same are administered to a subject on a fasted/empty stomach.

The resveratrol solubulization product formulations and oral pharmaceutical compositions described herein may be administered to a subject (e.g., human) in any suitable formulation according to conventional pharmaceutical practice (see, e.g., Remington: The Science and Practice of Pharmacy (21st ed.), ed. A. R. Gennaro, Lippincott Williams & Wilkins, (2005) and Encyclopedia of Pharmaceutical Technology, (3^{rd} ed.) eds. J. Swarbrick and J. C. Boylan, Marcel Dekker, CRC Press, New York (2006), a standard text in this field, and in USP/NF). A description of exemplary pharmaceutically acceptable carriers and diluents, as well as pharmaceutical formulations, can be found in Remington: supra. Typically, the resveratrol solubulization product formulations and oral pharmaceutical compositions described herein are administered as, for example, a soft gelatin capsule, a hard gelatin capsule, a soft gelatin-free capsule, or a hard gelatin-free capsule.

The therapeutic methods described herein in general include administration of a therapeutically effective amount of the resveratrol solubulization product formulations and oral pharmaceutical compositions described herein to a subject (e.g., human) in need thereof, particularly a human, under fasting conditions. Such treatment will be suitably administered to subjects, particularly humans, suffering from, having, susceptible to, or at risk for a disease, disorder, or symptom thereof (e.g., neuro inflammation, neurological damage, AD, mild TBI, concussion, Friedrich's Ataxia, etc.). Determination of those individuals “at risk” can be made by any objective or subjective determination by a diagnostic test or opinion of a subject or health care provider.

The methods described herein can further include detecting a state or condition of neurologic inflammation and disease (e.g., AD, mild TBI, concussion, Friedrich's Ataxia, etc.) in the subject, e.g., diagnosis. The detection is typically done prior to administering to the subject a resveratrol solubulization product formulation or oral pharmaceutical composition as described herein. Methods of detecting neurologic inflammation (e.g., oxidative stress, mitochondrial dysfunction) and associated disorders in a subject are well known in the art, and include detection of behavioral dysfunction, neurological deficits including cognitive, visual or auditory impairment, etc.

The methods can further include analyzing an endpoint (e.g., a therapeutic endpoint or marker) after administration of the resveratrol solubulization product formulation or oral pharmaceutical composition as described herein such as circulating plasma levels of resveratrol, and/or a blood concentration of resveratrol greater than 220 ng/ml (e.g., about 220 ng/ml to about 400 ng/ml, about 260 ng/ml to about 375 ng/ml, about 300 ng/ml to about 350 ng/ml) at Cₘₐₓ at a level of AUC below 2100 ng*hr/mL, and determining if the subject is or will be responsive. As additional examples, inflammation levels in the subject and lessening or elimination of the subject's disease symptom(s) can be measured to determine clinical outcomes or benefits.

### Effective Doses

The resveratrol solubulization product formulations as described herein and oral pharmaceutical compositions described herein are preferably administered to an individual in need thereof (e.g., human having neurological damage, and/or subjected to oxidative stress) in an effective amount, that is, an amount capable of producing a desirable result in a treated individual. Desirable results include one or more of, for example, decreasing or preventing neuroinflammation, oxidative stress and/or mitochondrial dysfunction, delaying onset of disease, decreasing or preventing neuronal death or damage, and prolonging cognitive abilities. Such a therapeutically effective amount can be determined according to standard methods. Toxicity and therapeutic efficacy of the resveratrol solubulization product formulations and oral pharmaceutical compositions as described herein utilized in the methods described herein can be determined by standard pharmaceutical procedures. As is well known in the medical and veterinary arts, dosage for any one individual depends on many factors, including the individual's size, body surface area, age, the particular composition to be administered, time and route of administration, general health, and other drugs being administered concurrently. A delivery dose of a resveratrol solubulization product formulation as described herein is determined based on preclinical and clinical efficacy and safety (e.g., see the safety results in the Examples below showing that oral administration of a resveratrol solubulization product formulation as described herein containing 500 mg of resveratrol is safe).

### EXAMPLES

The present invention is further illustrated by the following specific examples. The examples are provided for illustration only and should not be construed as limiting the scope of the invention in any way.

### Example 1—Resveratrol Solubulization Product Formulation Plasma PK Studies

PK studies with a single ascending dose (SAD) with food effect were conducted. The 3-leg ascending dose was conducted at 200, 500 and 700 mg; the group included subjects up to 75 years of age. The study also included a food effect cohort. Safety was evaluated by collecting any reports of adverse events. Resveratrol and 3 key metabolites (2 glucuronides and one sulfate) were measured in blood plasma and urine. Twenty-one subjects completed the first leg, 16 subjects completed the second leg, 18 subjects were dosed in the third leg, and 14 subjects completed the food effect leg (COVID-19 reduced enrollment). No treatment-emergent Serious Adverse Events (AEs) were observed at any dose. There was a limited number of possibly treatment-emergent minor AEs (mild headache, drowsiness). The Cₘₐₓ indicated 8 times higher bioavailability than standard resveratrol. The results also showed that bioavailability is not proportional; resveratrol levels actually increased disproportionally with increased dosing. The resveratrol solubulization product formulation as described herein achieved significantly improved bioavailability compared to resveratrol available in the nutritional market. Blood plasma Cₘₐₓ targets can be readily achieved for evaluation of efficacy (mean Cₘₐₓ above 220 ng/ml), and the plasma Cₘₐₓ targets can be achieved without approaching the FDA stipulated AUC limit (2100 ng*hr/ml). These PK studies are described in more detail below.

Period 1

Following single oral dose administration of JOTROL™ (resveratrol) 200 mg under fasting conditions, the following observations were made for resveratrol:

Resveratrol plasma concentrations were measurable up to 10 hours, with most subjects having between 3 to 6 measurable concentrations. Only 1 subject (Subject 11) had 2 measurable concentrations. The terminal elimination phase could not be properly characterized for 20 out of the 21 subjects due to the limited number of measurable resveratrol concentrations. Therefore, Kₑₗ, AUC_{0-inf} and T_{1/2} could only be determined for Subject 01.
Mean Cₘₐₓ value was 127.35 ng/mL, and was reached between 0.25 to 2 hours (median Tₘₐₓ of 1 hour).
Mean AUC₀₋ₜ was 148.40 h*ng/mL.
AUC_{0-inf} was 214.29 h*ng/mL; and the residual area was 5.48% (less than 20%)
T_{1/2} was 1.25 hours
Mean T_{1/2} was 2.52 hours

Conclusion:

As per protocol, the maximum threshold for resveratrol AUC exposures is 2100 h*ng/mL (equivalent to 2.1 h*μg/mL); i.e systemic exposure should not be greater than 2100 h*ng/mL. At a single oral dose of 200 mg resveratrol, JOTROL™ formulation achieves a total exposure of 214.29 h*ng/mL, which does not exceed the AUC threshold. Based on these results, the dose was escalated to the next dose level of 500 mg.

Period 2

Following single oral dose administration of JOTROL™ (resveratrol) 500 mg under fasting conditions, the following trends were observed for resveratrol:
Peak resveratrol plasma concentrations were achieved between 0.5 to 2 hours post-dose (median Tₘₐₓ of 1 hour).
   i. Concentrations were measurable between 0.133 and 12 hours.
   ii. All 16 subjects had concentrations values below the lower limit of quantification (BLQ) from 16 to 32 hours post-dose.
As observed on overlay plots, the variability among individuals was high with CV % on concentration data by timepoint within 22.51% to 141.47% for resveratrol, 30.39% to 128.4% for resveratrol-3-glucuronide, 24.38% to 134.07% for resveratrol-4-glucuronide and 29.66% to 116.28% for resveratrol sulfate.
The mean primary exposure PK parameters of resveratrol were 481.19 h*ng/mL, 576.23 h*ng/mL and 455.38 ng/mL for AUC₀₋ₜ, AUC_{0-inf}, and Cₘₐₓ, respectively. The variability (CV %) for AUCs and Cₘₐₓ was ˜53% to 90%.
   i. The mean residual area (or percentage of AUC extrapolated from the last measurable concentration up to infinity) was less than 20% for all subjects that had available residual area.
   ii. The mean T_{1/2} of resveratrol was 2.74 hours
The primary exposure PK parameters for resveratrol appeared to be approximately trending towards dose proportionality. A 2.5 fold increase in PK exposure parameters was expected with a dose increase from 200 mg to 500 mg resveratrol. These were the observed findings, which were slightly greater than 2.5 fold, based on the geometric means.
   iii. AUC₀₋ₜ increased 3.87 fold from Period 1 (200 mg) to Period 2 (500 mg), with an intra-subject variability of 60.70%.
   iv. Cₘₐₓ increased 3.63 fold from Period 1 (200 mg) to Period 2 (500 mg), with an intra-subject variability of 61.79%.
   v. Using the power model, approximate dose proportionality will be concluded if the 90% CI for the slope is entirely within the acceptance criterion (0.244, 1.756).
      The upper bound of the 90% confidence interval for AUC₀₋ₜ [90% CI=1.031, 1.898)], and Cₘₐₓ [90% CI=(0.865, 1.836)] fell outside the acceptance criterion. Based on these results, the dose proportionality criterion was not satisfied. However, with more data from the subsequent dose levels, the estimates are likely to change.
Based on the PK model predictions
   i. A dose of 700 mg was predicted to produce a mean AUC of 701 h*ng/mL, with a maximum exposure of 1578 h*ng/mL
   ii. A dose of 800 mg was predicted to produce a mean of AUC of 852 h*ng/mL, with a maximum exposure of 1919 h*ng/mL
   iii. A dose of 1000 mg was predicted to produce a mean of AUC of 1822 h*ng/mL, with a maximum exposure of 2660 h*ng/mL
   iv. A dose of 1140 mg was predicted to produce a mean AUC of 1432 with a maximum exposure of 2092 h*ng/mL in at least 90% of all subjects.

Conclusion
As per protocol, the maximum threshold for resveratrol AUC exposures is 2100 h*ng/mL (equivalent to 2.1 h*μg/mL); i.e systemic exposure should not be greater than 2100 h*ng/mL.
At a single oral dose of 500 mg resveratrol, JOTROL™ formulation achieved a mean exposure (AUC_{0-inf}=588.53 h*ng/mL with a range of 247.04 to 1030.80 h*ng/mL; AUC₀₋ₜ=486.60 h*ng/mL with a range of 216.08 to 989.10 h*ng/mL which does not exceed the AUC threshold.
Based on the PK model predictions, dose of 700 mg and 800 mg are likely to produce exposures below the AUC threshold (2100 h*ng/mL) for all subjects
All the predictions are based on limited number of observations from two periods only and the estimations should be used with precautions.
Upon review by the safety committee, 700 mg was recommended as the next dose level for Period 3.

Period 3

Following single oral dose administration of JOTROL™ (resveratrol) 700 mg under fasting conditions, the following trends were observed for resveratrol:
Peak resveratrol plasma concentrations were achieved between 0.25 to 2 hours post-dose (median Tₘₐₓ of 1 hour).
   i. Concentrations were measurable between 0.133 and 16 hours; except at 12 hours where all samples were below the lower limit of quantitation (BLQ).
   ii. All 18 subjects had concentrations values below the lower limit of quantification (BLQ) from 24 to 32 hours post-dose.
As observed on overlay plots, the variability among individuals was high with CV % on concentration data by timepoint within 26.5% to 174.98% for resveratrol.
Mean Cₘₐₓ was 805.44 ng/mL with variability of ˜73%.
The mean AUC₀₋ₜ was 892.86 h*ng/mL and ranged from 329.35 to 2181.56 h*ng/mL; variability was ˜56%.
   i. Of note Subject 22 had AUC₀₋ₜ (2181.56 h*ng/mL) above the AUC threshold of 2100 h*ng/mL
As the terminal elimination phase could only be adequately characterized for only 3 subjects, AUC_{0-inf} and T_{1/2} could only derived for those 3 subjects.
   i. Mean AUC_{0-inf} was 611 h*ng/mL; with variability (CV %), of ˜28%.
   ii. The mean residual area (or percentage of AUC extrapolated from the last measurable concentration up to infinity) was less than 20% for all subjects that had available residual area.
   iii. The mean T_{1/2} of resveratrol was 1.61 hours, and ranged from 1.15 to 1.93 hours
A 3.5 fold increase in dose from 200 mg to 700 mg produced a greater than proportional increase in resveratrol exposure. These were the observed findings, based on the ratio of geometric means for AUC and Cₘₐₓ.
   i. AUC₀₋ₜ increased 6.77 fold from Period 1 (200 mg) to Period 3 (700 mg), with an intra-subject variability of 68.67%.
   ii. Cₘₐₓ increased 6.78 fold from Period 1 (200 mg) to Period 3 (700 mg), with an intra-subject variability of 70.67%.
Further, using the power model, approximate dose proportionality would be concluded if the 90% CI for the slope falls entirely within the acceptance criterion (0.447, 1.553).
   i. The upper bound of the 90% confidence interval for AUC₀₋ₜ [90% CI=1.264, 1.798)], and Cₘₐₓ [90% CI=(1.186, 1.806)] falls outside the acceptance limits for conclusion of dose proportionality, indicating that within the dose range of 200 mg to 700 mg, exposure increases disproportionately (greater than proportional) with dose.
Model Performance for PK predictions of Exposure at 700 mg
   i. Based on 37 subjects (from Period 1 and Period 2), a dose of 700 mg was predicted to produce a mean of AUC of 701 h*ng/mL, with a maximum exposure of 1578 h*ng/mL.
   ii. It is important to point out that due to the small sample size used in the model, these predictions were made cautiously and limited to the 37 subjects from periods 1 and 2.
   iii. The model performance was very good in regards to all subjects, mentioned above, who were crossed over from previous periods to Period 3. The maximum AUC₀₋ₜ observed for these subjects was 1685.80 h*ng/mL (predicted 1578 h*ng/mL).
   iv. Three new subjects (22, 23 and 24), however, were enrolled in period 3; meaning data were not available for them from previous periods. Subject 22 had AUC of 2181.56 h*ng/mL in Period 3, it is likely that this subject would have had higher exposures with the previous dose levels (200 mg and 500 mg) as well, and addition of this data would further improve the model predictions.

Conclusion
As per protocol, the maximum threshold for resveratrol AUC exposures is 2100 h*ng/mL (equivalent to 2.1 h*μg/mL); i.e. systemic exposure should not be greater than 2100 h*ng/mL.
At a single oral dose of 700 mg resveratrol, JOTROL™ formulation achieved a mean exposure (AUC₀₋ₜ=892.86 h*ng/mL and ranged from 329.35 to 2181.56 h*ng/mL; AUC_{0-inf}=611 h*ng/mL h*ng/mL with a range of 473.36 to 803.91 h*ng/mL.
Given that a dose of 700 mg produced an AUC level >2100 h*ng/mL in one subject (Subject 22), and the effect of food on the absorption of resveratrol from the JOTROL™ is unknown, we suggest that a lower dose (500 mg) be used for the food effect study.

Food Effect—Period 4 vs Period 2

Following single oral dose administration of JOTROL™ (resveratrol) 500 mg under fasting and fed conditions, the following trends were observed for resveratrol.
The absorption of resveratrol was slightly slower under fed conditions a median peak concentration observed 1.50 hours post-dose compared to 1.00 hours post-dose, hence a delay of 0.5 hours was noted. Overall, the Tmax, across both fed and fasted conditions, ranged from 0.500 to 2.00 hours.

The comparison between fed (Treatment D) and fasted (Treatment B) treatment groups was performed upon the two-sided 90% CIs for the ratios of the geometric means (GMR) (fed/fasted) using a no-food effect acceptance criteria of 80.00-125.00%.
The GMR (fed/fasted) and 90% CI were:
i. 56.92% (42.04% to 77.07%) for AUC0-t
ii. 41.91% (24.52% to 71.63%) for Cmax
The intra-subject variability was about 79% for Cmax and 41% for AUC0-t.
Based on the ratio (Fed/Fasting) and 90% CI for AUC0-t, and Cmax, the extent (AUC) and rate (Cₘₐₓ) of absorption of resveratrol following administration of a 500 mg JOTROL™ dose under fed conditions is about 57% and 42%, respectively, of that observed under fasting conditions, (˜43% lower AUC and 58% lower Cmax).

### Example 2—Comparison of Resveratrol Solubulization Product Formulation to Nutritional Resveratrol in Friedreich's Ataxia and AD Studies

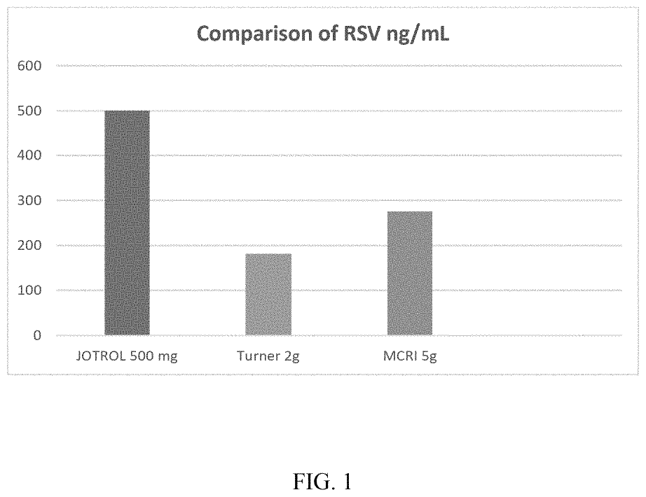

Referring to FIG. 1, the PK studies of Example 1 show that administration of a resveratrol solubulization product formulation as described herein increased bioavailability of resveratrol compared to nutritional resveratrol (MCRI) administered at 2.5 grams resveratrol twice daily in a Friedreich's Ataxia study (‘MCRI 5g’—Yiu et al., J Neurol. 2015; 262(5):1344-53), and compared to MCRI administered at 1.0 gram resveratrol twice daily in an AD study (‘Turner 2g’—Turner et al., Neurology 2015; 85: 1383-1391). 300 ng/ml at C-Max is seen as a minimum to achieve therapeutic effect.

### Example 3—Safety and Pharmacokinetics of a Highly Bioavailable Resveratrol Preparation (JOTROL™)

This Example describes a first in human study (FIH) to evaluate the bioavailability of resveratrol after ascending, single oral doses up to 700 mg resveratrol as JOTROL™. After a single 500 mg dose of JOTROL™, a Cmax of 455 ng/mL was observed, vs. 85 ng/mL Cmax after a 1 g encapsulated dose (Turner et al., Neurology 85:1383-91, 2015) and 1942 ng/mL after a 2.5 g micronized dose (Howells et al., Cancer Prev Res (Phila) 4:1419-1425, 2011). In this study, resveratrol exposures (AUCs and Cmax) increased with increasing doses. This increase appears to be higher than dose-proportional for AUC₀₋ₜ and Cmax. Resveratrol and its three major conjugates (JOTROL™) accounted for 40 to 55% of the dose in urine, consistent with a high extent of absorption, but <1% of drug-related material was intact relative to key metabolites in plasma and urine.

The objectives of this PK study were to characterize the PK profile of JOTROL™ (with resveratrol API) following oral administration of single ascending doses (SAD) ranging from 200 mg up to a dose estimated to be 1000 mg in healthy adult subjects. In addition, the effect of food on the PK profile of JOTROL™ was also determined. Dosage levels were based on the plasma levels achieved by the previously reported population PK data and the attainment of target plasma levels at Cmax while not exceeding an AUC level of 2100 ng*h/mL as per discussions with the US Food and Drug Administration. Based on the data from the first 2 study periods, the third period dose was reduced by the SRC to 700 mg and 500 mg was used in the fourth, fed period. The study also evaluated safety and tolerability. The use of resveratrol as JOTROL™ in MPS 1, Friedreich's ataxia, MELAS, traumatic brain injury, and AD/mild cognitive impairment, is being pursued, among other indications.

Formulation and Drug Product Development

Details of the JOTROL™ formulation (also referred to herein as “resveratrol solubulization product” and “solubilization product”) are found in U.S. Pat. No. 10,780,056. For production of 1 kg of the solubilization product, 100 g resveratrol; 45 g medium-chain triglycerides; 600 g polysorbate 80; 180 g polysorbate 20, and 75 g mixed tocopherols were used. To protect trans-resveratrol from light degradation, all manufacturing and testing procedures steps were conducted under yellow light.

The resveratrol was (trans-)resveratrol, 99%, CAS number 501-36-0, procured from Evolva, Reinach Switzerland. The CAS number is an international reference standard for chemical substance. Each known chemical substance has a unique CAS number. Medium chain triglyceride (MCT) oil (70/30) Rofetan GTCC 70/30 made by DHW Deutsche Hydrierwerke Rodleben GmbH, Dessau-RoJlau, Germany, CAS number 73-398-61-5 was used as the medium-chain triglycerides. Commercial preparations, for example, TEGO SMO 80 V, Evonik or Crillet 4/Tween 80-LQ-(SG), Croda GmbH, Nettetal, Germany, can be used as polysorbate 80 (E433, CAS number 9005-65-6). Commercial preparations, for example, TEGO SML 20 V, Evonik or Crillet 1/Tween 20-LQ-(SG), Croda GmbH, Nettetal, Germany, can be used as polysorbate 20 (E432, CAS number 9005-64-5). Vitapherole T-70 Non GMO, a 70% mixed tocopherols in plant oil made by Vitae Caps S.A., Spain, or EMix 70 made by Nutrilo GmbH, Cuxhaven, Germany, can be used as mixed tocopherols (E306, CAS numbers 59-02-9, 16698-35-4, 54-28-4, and 119-13-1). Polysorbate 20, polysorbate 80, mixed tocopherols, and MCT oil were homogenized at a temperature in the range of approximately 18° C. to approximately 22° C. while stirring.

Preclinical Evaluation

Circulating plasma levels of resveratrol in the formulations as described herein have shown a surprisingly high level of circulating resveratrol compared to native resveratrol delivered as suspension or delivery of micronized resveratrol API as a suspension as tested in mice and rats. A 5% resveratrol solubilization product-based formula prototype of JOTROL™ (which is 10% dose loaded) show a higher maximal plasma drug level (“Cmax”) than resveratrol API from the same source and micronized resveratrol from another source. The JOTROL™) prototype formulation also showed a higher total absorption amount (the “AUC”). A study in rats at a surprisingly high 10% dose loading showed similarly high plasma levels of resveratrol. These test results are summarized.

Mice were tested for relative plasma bioavailability of resveratrol administered orally from different formulations. At 50 mg/kg resveratrol delivered as the 5% dose loaded solubilization product, the Cmax was 17 fold higher than with unformulated API and more than 10 fold higher than micronized Mega Resveratrol. Micronized resveratrol showed slightly higher absorption than standard API. When treated at 25 mg/kg, the solubilization product group showed less than half the resveratrol absorption than the 50 mg/kg dose of the solubilization product but was broadly similar to the 50 mg/kg standard suspension treatments.

The AUC (for 4 h after dosing with 50 mg/kg) was 4 fold higher for the solubilization product than for the Micronized Mega Resveratrol. In rats, the 10% dose loaded solubilization product dosed at 50 mg/kg showed a Cmax was 7 fold higher than the level observed with micronized resveratrol, and the AUC (for 24 h after dosing) was two and a half fold higher for the solubilization product than for Micronized Mega Resveratrol.

The terminal elimination rate of resveratrol from the solubilization product or from Micronized Mega Resveratrol was the same and consists with literature, meaning that the solubilization product formula does not alter resveratrol metabolism after resveratrol is present in plasma.

In summary, the solubilization product forms of orally administered resveratrol (JOTROL™) offer superior absorption properties as compared to standard forms. The resveratrol solubilization product formulas clearly outperformed the non-micellar dosing form. Inter-species dose scaling is consistent with expectations, suggesting a dose reduction exploiting the solubilization product is achievable in man.

Pharmacokinetic Study Considerations and Design

A total of 24 healthy, adult male or female volunteers were included in study part 1. In part 1, all subjects were sequentially dosed under fasting conditions in an ascending manner across 3 dose levels (200 mg, 500 mg, and 700 mg). A food effect arm was also included as part 2. Key design parameters are summarized in Table 1. While resveratrol is an OTC product, this was the FIE study with the JOTROL™ formulation. As such, the PK of resveratrol in JOTROL™ were unknown and a reasonable estimate of subject number needed was not evaluable.

TABLE 1
Study drug formulation and test cohorts

| | Study period 1 | Study period 2 | Study period 3 | Study period 4 |
|---|---|---|---|---|
| | (treatment A) | (treatment B) | (treatment C) | (treatment D) |
| Product | JOTROL ™ | JOTROL ™ | JOTROL ™ | JOTROL ™ |
| | (resveratrol) gelcaps | (resveratrol) gelcaps | (resveratrol) gelcaps | (resveratrol) gelcaps |
| Treatment | A | B | C | D |
| code | | | | |
| Strength | 100 mg | 100 mg | 100 mg | 100 mg |
| Dosage | 2 × 100 mg gelcaps | 5 × 100 mg gelcaps | 7 × 100 mg gelcaps | 5 × 100 mg gelcaps |
| form | | | | |
| Dose | 200 mg | 500 mg | 700 mg | 500 mg |
| administered | | | | |
| Route of | Oral; fasting | Oral; fasting | Oral; fasting | Oral; fed |
| administration | | | | |

| | |
|---|---|
| Inactive | Polysorbate 80, polysorbate 20, mixed tocopherols concentrate, |
| ingredients | fractionated coconut oil, triglycerides (medium chain) |
| Manufacturer | Catalent Pharma Solutions, St Petersburg, FL 33716-1016 |

A total of 4 study periods were included in this study with washout of at least 14 days between doses. In each study period, subjects were confined to the Syneos Health Clinical Research Facility from day 1 until after the 32-h post-dose blood draw. After the completion of each cohort, an evaluation of the safety data was performed prior to determining whether to proceed with enrollment for the next scheduled dose level, to modify the dose, or to discontinue the study.

The FDA preliminary review of the published data on resveratrol found that the nonclinical data could only support daily oral doses of unmodified resveratrol (i.e., non-micronized drug with no absorption enhancers) of up to 3000 mg/day for no more than 13 weeks. Published data from a 26-week study in mice Aaps Open demonstrated renal toxicity at 1000 mg/kg/day, for which the human equivalent dose is approximately 5000 mg. Based on published data in healthy volunteers, AUC exposure at a dose of 3,000 mg/day of unmodified resveratrol was estimated to be 2100 ng·h/mL. Because JOTROL™ formulation has been modified to enhance bioavailability, JOTROL™ doses to be used in this study were to maintain AUC exposures below 2100 ng·h/mL.

Study part 1 (periods 1, 2, and 3): No food was allowed from at least 10 hours before dosing until at least 4 hours after dosing.

Study part 2 (period 4): After a supervised fast of at least 11 hours (h), subjects were served a critical, high-fat, high-calorie meal of approximately 800 to 1000 calories (approximately 50% of total caloric content of the meal derived from fat). Drug administration occurred 30±1 m after the meal has been started.

Meals were standardized and similar in composition between periods. Except for fluids provided with the critical breakfast (study part 2 only) and water given with study medication, no fluids were allowed from 1 h before dosing until 1 h post-dose. Water was provided ad libitum at all other times.

Sample collection and processing: A saline intravenous catheter was used for blood collection to avoid multiple skin punctures. Otherwise, blood samples were collected by direct venipuncture. The total volume of blood drawn from each subject completing this study did not exceed 400 mL.

Blood samples: All blood samples were drawn into blood collection tubes (17×3 mL) containing dipotassium ethylenediaminetetraacetic acid (K₂EDTA) prior to drug administration and 0.133, 0.250, 0.500, 1.00, 1.50, 2.00, 3.00, 4.00, 5.00, 6.00, 8.00, 10.0, 12.0, 16.0, 24.0, and 32.0 h post-dose, during each period. Sample collections done outside the pre-defined time windows (±1 min for samples collected before 8 h post-dose and ±3 min for subsequent samples) were not considered as protocol deviations since actual post-dose sampling times are used for PK and statistical analyses. Blood samples were cooled in an ice bath and were centrifuged at 2000±5×g for at least 10 min at approximately 4° C. (no more than 240 min passed between the time of each blood draw and the start of centrifugation). Two aliquots of at least 0.5 mL (when possible) of plasma were dispensed into polypropylene tubes as soon as possible. The aliquots were transferred to a −80° C. freezer (no more than 60 min passed between the start of centrifugation and aliquot storage), pending analysis/shipment to the analytical facility.

Since resveratrol is sensitive to UV light, blood and plasma collection tubes were protected from light, sample processing was performed under sodium lamp or yellow/gold light, and samples were transferred into amber vials. At the end of the study, all samples were transferred to the bioanalytical facility (Syneos Health, Princeton, N.J.). All transfers between sites were sent in two shipments: one for each set of aliquots. Frozen plasma aliquots were sent with sufficient dry ice to maintain the aliquots in a frozen state for at least 72 h.

Urine samples: Urine samples were collected and pooled according to the following intervals: pre-dose (within 2 h before dosing), 0-4 h, 4-8 h, 8-12 h, 12-24 h, and 24-32 h post-dose. For day 2 (24 h post-dose) urine collection, subjects were asked to void their bladder within 15 min before the end of the collection interval (12-24 h). For other collections, subjects were asked to void their bladder within 10 min before the end of each collection interval. Urine voided at the intersection of two intervals was included in the earlier interval. Any urine voided by subjects but not collected was documented. The volume of urine collected in each interval was measured (individual urine volumes are on file), and two aliquots of equal volume were dispensed into polypropylene tubes for each interval. Aliquots were stored in a −80° C. freezer, pending analysis; remaining urine from each subject was discarded.

Urine collection tubes were protected from UV light, sample processing was performed under sodium lamp or yellow/gold light, and samples were transferred into amber vials. At the end of the study, the first set of frozen urine aliquots from the clinical facility, accompanied by an inventory list and sufficient dry ice to maintain the aliquots in a frozen state for at least 72 h, were sent to the bioanalytical facility (Syneos Health, Princeton, N.J.). Bioanalytical methods for detection and quantitation of resveratrol, resveratrol sulfate, resveratrol 3-glucuronide, and resveratrol 4-glucuronide in human plasma K2EDTA and human urine have been validated in compliance with the FDA 2018 Bioanalytical Method Validation Guidance for Industry. The method for human plasma has been proven to be precise, accurate, sensitive, and selective over the concentration range studied (5.00 to 5000 ng/mL for resveratrol and resveratrol sulfate and 2.00 to 2000 ng/mL for resveratrol 3-glucuronide and resveratrol 4-glucuronide). Samples are extracted by a protein precipitation extraction procedure, and the compounds are detected and quantified by tandem mass spectrometry in positive ion mode on an MDS Sciex API 6500+ equipped with a Turbo Ionspray® interface. Incurred sample reproducibility was within FDA guidance acceptance criteria for all compounds using this assay.

The method for human urine was proven to be precise, accurate, sensitive, and selective over the concentration range studied (5.00 to 5000 ng/mL for resveratrol, 100 to 100,000 ng/mL for resveratrol sulfate, and 20.0 to 20000 ng/mL for resveratrol 3-glucuronide and resveratrol 4-glucuronide). Samples are extracted by a dilution extraction procedure, and the compounds are detected and quantified by tandem mass spectrometry in positive ion mode on an MDS Sciex API 6500+(or an MDS Sciex API 4000 for resveratrol sulfate) equipped with a Turbo Ionspray® interface. Incurred sample reproducibility was within FDA guidance acceptance criteria for all compounds using this assay. All concentration values that were below the lower limit of quantification (BLQ) occurring prior to dosing as well as samples with no reportable value (NRV) occurring prior to dosing were replaced by “0.00”; otherwise, they (BLQ and NRV) were set to missing for tabulation, graphical representation, and calculation purposes. PK analyses were performed using Phoenix WinNonlin® version 8.2, which was validated by Syneos Health. The WinNonlin noncompartmental analysis (NCA) module was used to calculate AUC₀₋ₜ (last detectable concentration), AUC_{0-inf} (infinity), residual area (%), Cmax, Tmax, T½ el (elimination half-life), and Kel (elimination rate constant). Dose proportionality analysis for AUC₀₋ₜ, AUC_{0-inf} and Cmax was performed (using the power model with mixed procedure from SAS®) considering data under fasting conditions (periods 1, 2, and 3). Power model included the PK parameter as the response variable and dose (mg) as the explanatory variable. For this model, the variable dose was treated as a continuous variable. For evaluation of the food-effect, PK data (1n-transformed AUC₀₋ₜ, AUC_{0-inf}, Cmax and untransformed Tmax) reported under fed conditions (Period 4) and under fasting conditions (for the same dose level) were compared using analysis of variance (ANOVA) from SAS®. The ratio (fed/fasting) and 90% geometric CI were also calculated for AUC₀₋ₜ, AUC_{0-inf}, and Cₘₐₓ.

Results

The safety population consisted of 24 subjects who received at least one dose of study medication (JOTROL™). Fourteen (14) subjects completed the study, and 10 subjects were discontinued. Of these, 11 subjects completed all 4 treatment periods (treatments A, B, C and D) and received all planned doses, namely, subject nos. 01, 05, 06, 08, 10, 11, 13, 15, 16, 17, and 19. In part 1 of the study, fifteen (15) subjects received all 3 treatments (treatments A, B, and C), namely, subject nos. 01, 05, 06, 07, 08, 09, 10, 11, 13, 15, 16, 17, 19, 20, and 21. Of the 24 subjects included in part 1 of the study, a total of 15 subjects (subject nos. 01, 03, 05, 06, 08, 10-13, 15-17, 19, 23, and 24) were enrolled in part 2. Of the 15 subjects enrolled, 14 (93.3%) subjects completed the period 4. The following subjects received some, but not all, planned treatments:
Subject nos. 02, 04, 14, and 18 received only treatment A and did not receive treatments B and C
Subject no. 03 received only treatments A and D and did not receive treatments B and C
Subject no. 12 received only treatment A, B, and D and did not receive treatment C
Subject no. 22 received only treatment C and subject nos. 23 and 24 received only treatment C and D. All these subjects did not receive treatments A and B

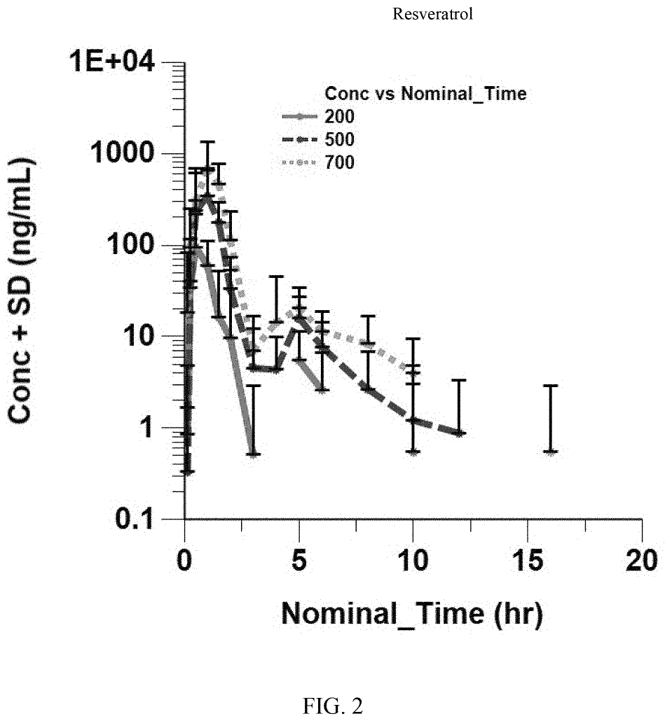

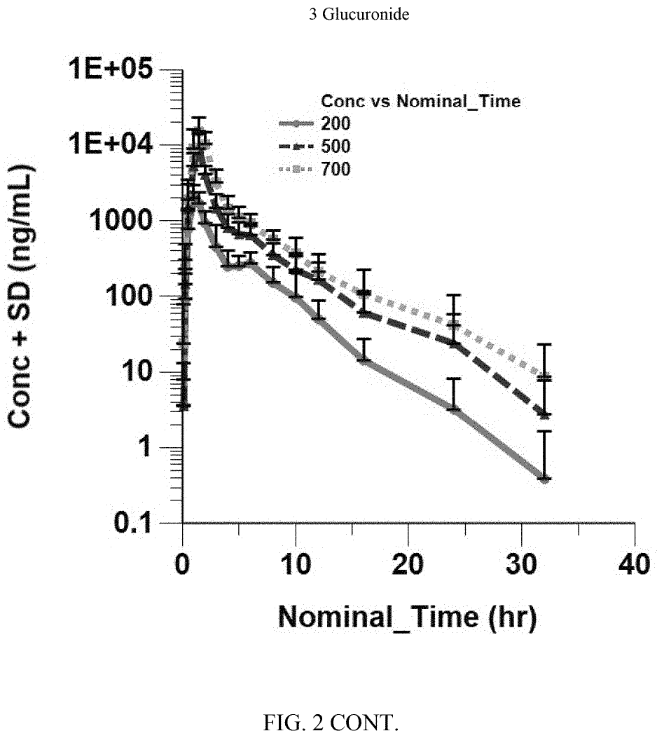

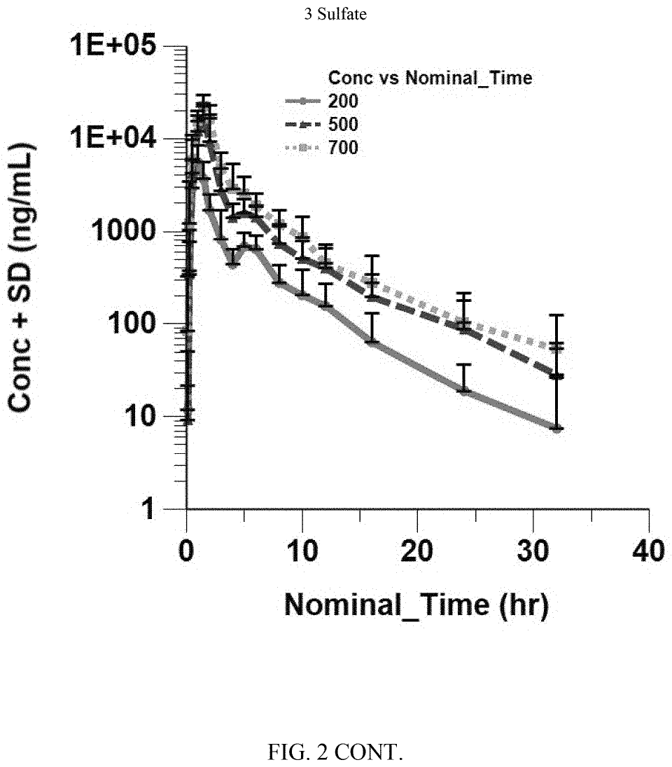

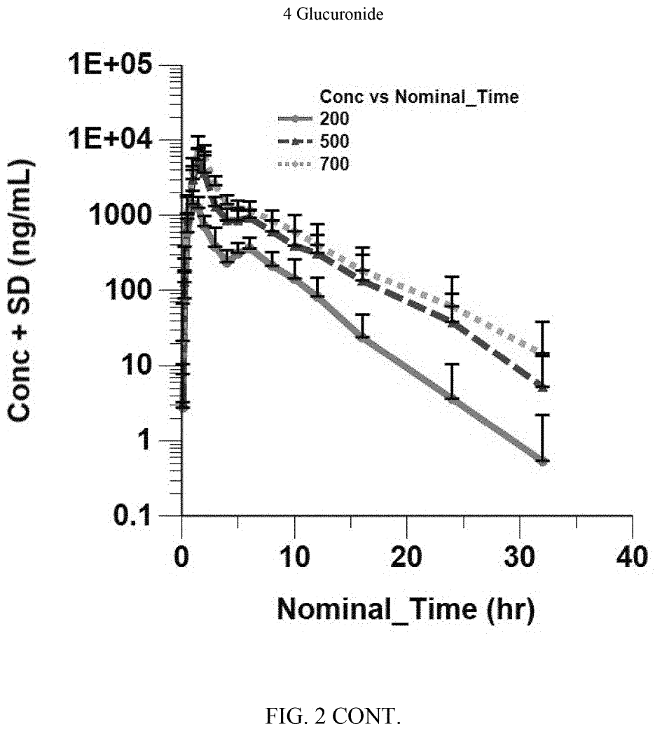

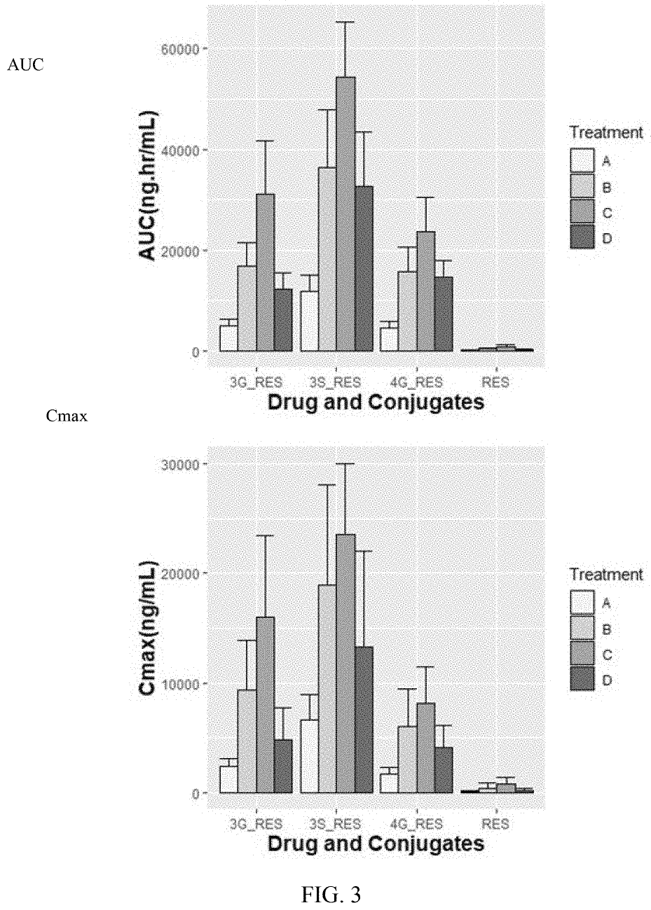

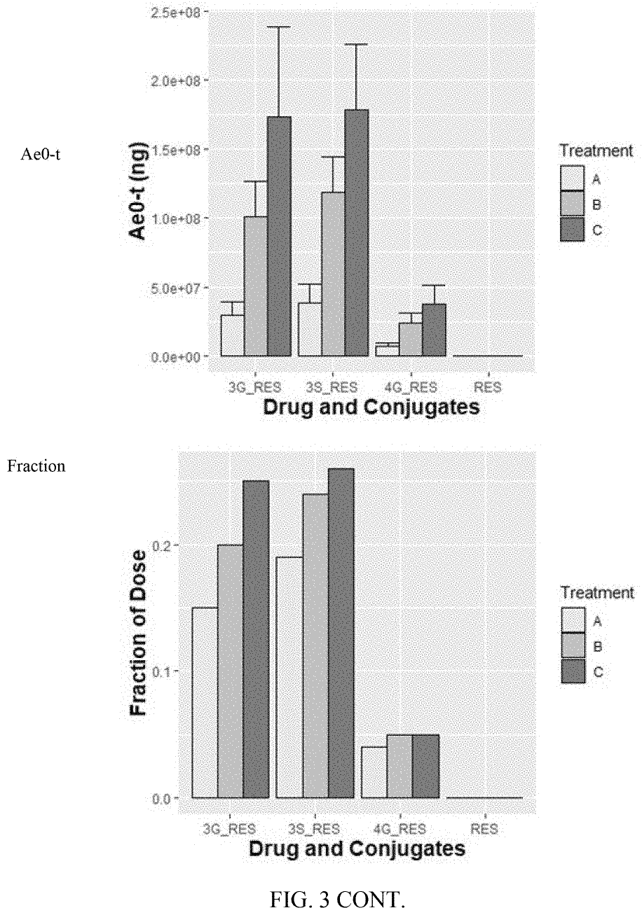

Plasma concentrations are summarized in FIG. 2. PK parameters are summarized in Table 2 and compared in FIG. 3.

TABLE 2
Summary of plasma pharmacokinetic parameters of JOTROL ™ (resveratrol) (PK population)

| | | Treatment A | | | | Treatment B | | | |
|---|---|---|---|---|---|---|---|---|---|
| Analyte | Parameter (unit) | N | Mean | SD | CV % | N | Mean | SD | CV % |
| Resveratrol | AUC₀₋ₜ (h*ng/mL) | 21 | 149 | 108 | 72 | 16 | 480 | 256 | 53 |
| Plasma | AUC_{0-inf} (h*ng/mL) | 1 | 215 | — | — | 5 | 574 | 325 | 57 |
| | Residual Area (%) | 1 | 5.48 | — | — | 5 | 5.55 | 4.08 | 74 |
| | Cₘₐₓ (ng/mL) | 21 | 127 | 116 | 91 | 16 | 455 | 409 | 90 |
| | T_{1/2 el} (h) | 1 | 1.26 | — | — | 5 | 2.74 | 1.71 | 63 |
| | Kₑₗ (/h) | 1 | 0.552 | — | — | 5 | 0.332 | 0.162 | 49 |

| | | Treatment A | | | | Treatment B | | | |
|---|---|---|---|---|---|---|---|---|---|
| Analyte | Parameter (unit) | N | Median | Min | Max | N | Median | Min | Max |
| Resveratrol | Tₘₐₓ (h) | 21 | 0.999 | 0.25 | 2.01 | 16 | 1 | 0.496 | 2.001 |

| | | Treatment A | | | | Treatment B | | | |
|---|---|---|---|---|---|---|---|---|---|
| Analyte | Parameter (unit) | N | Mean | SD | CV % | N | Mean | SD | CV % |
| Urine | Ae0-t (ng) | 21 | 22700 | 17100 | 75 | 16 | 54300 | 27100 | 50 |
| | Rmax (ng/h) | 21 | 12100 | 10000 | 82 | 16 | 39300 | 53700 | 137 |
| | TRmax (h) | 21 | 1.5 | 2.49 | 166 | 16 | 0.678 | 0.384 | 57 |
| | CLR (L/h) | 21 | 0.25 | 0.28 | 112 | 16 | 0.14 | 0.09 | 63 |

| | |
|---|---|
| Treatment C | Treatment D |

| Analyte | Parameter (unit) | N | Mean | SD | CV % | N | Mean | SD | CV % |
|---|---|---|---|---|---|---|---|---|---|
| Resveratrol | AUC₀₋ₜ (h*ng/mL) | 18 | 886 | 456 | 52 | 14 | 306 | 201 | 66 |
| Plasma | AUC_{0-inf} (h*ng/mL) | 3 | 611 | 172 | 28 | 2 | 613 | 373 | 61 |
| | Residual Area (%) | 3 | 3.76 | 1.45 | 39 | 2 | 3.7 | 4.12 | 111 |
| | Cₘₐₓ (ng/mL) | 18 | 805 | 590 | 73 | 14 | 205 | 207 | 101 |
| | T_{1/2 el} (h) | 3 | 1.6 | 0.4 | 25 | 2 | 1.71 | 1.22 | 72 |
| | Kₑₗ (/h) | 3 | 0.455 | 0.13 | 29 | 2 | 0.547 | 0.392 | 72 |

| | | Treatment C | | | | Treatment D | | | |
|---|---|---|---|---|---|---|---|---|---|
| Analyte | Parameter (unit) | N | Median | Min | Max | N | Median | Min | Max |
| Resveratrol | Tₘₐₓ (h) | 18 | 1.025 | 0.251 | 2 | 14 | 1.497 | 0.499 | 2.002 |

Treatment C

| Analyte | Parameter (unit) | N | Mean | SD | CV % |
|---|---|---|---|---|---|
| Urine | Ae0-t (ng) | 18 | 143000 | 127000 | 88 |
| | Rmax (ng/h) | 18 | 63400 | 57700 | 91 |
| | TRmax (h) | 18 | 0.974 | 0.796 | 82 |
| | CLR (L/h) | 18 | 0.19 | 0.18 | 95 |

No urinary data collected in treatment D. N = number of observations; SD = standard deviation; CV = coefficient of variation; Min = minimum; Max = maximum.

The absorption of resveratrol was similar for all tested doses after a fast, with Tmax values ˜1 h after dosing. After a meal, the Tmax increased to ˜1.5 h. Mean AUC0-t increased with increasing resveratrol doses, with mean values ranging from 149 to 886 h*ng/mL, while the mean Cmax ranged from 127 to 805 ng/mL. Using a power model to test for dose linearity, the increase in exposure was higher than expected if dose proportional. There are gender differences in the exposure of resveratrol (p=0.011). The mean female AUC values tend to be higher than male values at all doses. After a 500 mg dose and high fat meal, the mean AUC₀₋ₜ decreased from the value for a 500 mg dose after a fast, with mean value of 306 (vs 480) h*ng/mL and the mean Cmax of 205 (vs 455) ng/mL. This study demonstrates that the consumption of food prior to dosing affects the PK of resveratrol by lowering the rate and extent of absorption as the 90% geometric CI obtained for AUC₀₋ₜ and Cmax were not within the acceptance range and delayed the peak concentration by approximately 30 min. Relative to the three conjugates, resveratrol plasma concentrations were very low and the drug was eliminated rapidly, with a T½ of ˜2 h. The T½ values (as well as parameters also derived from the elimination rate constant) listed above should be interpreted with caution as N was low for most of the groups when calculating AUC_{0-inf}, T½, and Kel. This was due (mostly) to later timepoints falling below the lower limit of quantification. Renal clearance was low, ranging from 0.14 to 0.25 L/h (˜4 mL/min) and was similar between all treatments indicating a non-renal elimination pathway.

The median Tmax of resveratrol-3-glucuronide was similar for all tested doses ranging from 1.006 to 1.505 h post-dose. The mean resveratrol-3-glucuronide AUC₀₋ₜ increased with increase in resveratrol doses with mean values ranging from 4970 to 31000 h*ng/mL and while the mean Cmax ranged from 2390 to 16000 ng/mL. The median Tmax of resveratrol-4′-glucuronide was similar for all tested doses, ranging from 1.01 to 1.51 h post-dose. The mean resveratrol-4′-glucuronide AUC₀₋ₜ increased with increase in resveratrol doses, with mean values ranging from 4700 to 23600 h*ng/mL, and the mean Cmax ranged from 1710 to 8190 ng/mL The median Tmax of resveratrol-3-sulfate was similar for all tested doses ranging from 1.00 to 1.50 h post-dose. The mean resveratrol-3-sulfate AUC₀₋ₜ increased with increasing resveratrol doses, with mean values ranging from 12000 to 54500 h*ng/mL, while the mean Cmax ranged from 6620 to 23600 ng/mL. These three metabolites account for 40 to 55% of the total dose. Food consumption significantly decreased the concentrations for resveratrol-3-sulfate and resveratrol-3-glucuronide but not resveratrol-4′-glucuronide.

A total of 25 treatment emergent adverse events (TEAEs) were reported by 15 (62.5%) of the 24 subjects who received any amount of study drug. Nine (9) TEAEs were reported by 7 (33.3%) of the 21 subjects who received treatment A, 4 TEAEs were reported by 4 (25.0%) of the 16 subjects who received treatment B, 6 TEAEs were reported by 6 (33.3%) of the 18 subjects who received treatment C, and 6 TEAEs were reported by 6 (40.0%) of the 15 subjects who received treatment D. No clear trend was observed with number of TEAEs reported with increasing doses of resveratrol. Although the number of TEAEs were not notably different between treatment B (fasting) and treatment D (fed), there was a slight difference in the proportion of subjects who reported TEAEs. Around half (13/25; 52%) of all TEAEs were related to the study drug. There were no deaths, serious, or severe TEAEs reported. Most (23) TEAEs reported were mild in severity. The most commonly reported TEAEs during this study were in the SOC of nervous system disorders. The severity of TEAEs was graded according to the following categories: mild, moderate, or severe. Of the 25 TEAEs reported, 23 were graded as mild, and 2 were graded as moderate. None were graded as severe. Moderate TEAEs were reported by 1 subject (subject no. 02) after receiving treatment A (COVID-19) and 1 subject (subject no. 15) after receiving treatment C (syncope). Both these moderate TEAEs were recovered/resolved by the end of the study. The PI or a medical sub-investigator judged the relationship of each TEAE to the study medication using the following categories: unrelated (not related), possible, probable, and remote. Overall, of the 25 TEAEs reported, the relationship of 13 TEAEs was judged as possibly related, 8 as remotely related, and 4 as unrelated. Somnolence (6 [25%] subjects; 8 events) and headache (4 [16.7%] subjects; 4 events) were the most frequent events judged to be possibly related.

In conclusion, as the target level mean Cmax of 300 ng/ml (or more) in blood plasma resveratrol was achieved without approaching an upper limit AUC of 2100 ng*h/ml, the objective of identifying an obtainable and suitable/well-tolerated dose of JOTROL™ was achieved.

### Example 4—JOTROL™, a Novel Formulation of Resveratrol, Shows Beneficial Effects in the 3×Tg-AD Mouse Model

AD has minimally effective treatments currently. High concentrations of resveratrol, a polyphenol antioxidant found in plants, have been reported to affect several AD-related and neuroprotective genes. To address the low bioavailability of resveratrol and the negative side-effects of high dose resveratrol, JOTROL™ was investigated and was shown to have increased pharmacokinetic properties compared to non-formulated resveratrol in animals and in humans. To test if equivalent doses of JOTROL™, compared to non-formulated resveratrol, would result in greater brain exposure to resveratrol, and more efficacious responses on AD biomarker, for sub-chronic reversal studies, 15-month-old male triple transgenic (APPSW/PS1M146V/TauP301L; 3×Tg-AD) AD mice were treated orally with vehicle or 50 mg/kg JOTROL™ for 36 days. For prophylactic studies, male and female 3×Tg-AD mice were similarly administered vehicle, 50 mg/kg JOTROL™, or 50 mg/kg resveratrol for 9 months starting at 4 months of age. A behavioral battery was run, and mRNA and protein from brain and blood were analyzed for changes in AD-related gene and protein expression. The results showed that JOTROL™ displays significantly increased bioavailability over non-formulated resveratrol. Treatment with JOTROL™ resulted in AD-related gene expression changes (Adam10, Bace1, Bdnf, Psen1) some of which were brain region-dependent and sex-specific, as well as changes in inflammatory gene and cytokine levels. JOTROL™ can be used as a prophylaxis and/or treatment for AD through increased expression and/or activation of neuroprotective genes, suppression of pro-inflammatory genes, and regulation of central and peripheral cytokine levels.

Described here are the results of a completed Phase I clinical trial where no serious adverse effects were observed, suggesting feasibility for use of JOTROL™ as a therapeutic (ClinicalTrials.gov Identifier: NCT04668274). The results show that JOTROL™ has superior bioavailability in rodent models, and the effects of acute and long-term treatment with JOTROL in the triple-transgenic AD (3×Tg-AD) mouse model are reported.

### Materials and Methods

Animals and Treatment

Male and female triple-transgenic AD (3×Tg-AD) mice were purchased through The Jackson Laboratory and the NIH-supported Mutant Mouse Regional Resources Center (MMRRC). The 3×Tg-AD mouse overexpresses three human transgenes: the APP Swedish double mutation KM670/671NL (APPSW), the presenilin-1 M146V mutation (PS1M146V), and the TauP301L mutation. The 3×Tg-AD mouse is one of the few comprehensive AD models that present both human Aβ plaques and tau tangles (Oddo et al., Neurobiol Aging 24, 1063-1070). Mice were housed four animals per cage under a regular 12-h/12-h light/dark cycle and had ad libitum access to food and water, in a humidity- and temperature-controlled, AAALAC-accredited ani-mal facility at the University of Miami Miller School of Medicine.

For subchronic disease-reversal studies, 14-month-old male 3×Tg-AD were treated for 36 days orally with 50 mg/kg JOTROL™ or vehicle (water, 18% Tween 80; n=5/group). For long-term prophylactic studies, a cohort of 30 mice was used (50% males and females). Three groups of 10 (5 males and 5 females) were treated by oral gavage with either vehicle (saline, 5% Tween 80, 4.6% PEG 400), 50 mg/kg JOTROL™, or 50 mg/kg unformulated resveratrol (RSV) diluted in vehicle. JOTROL™ and RSV were prepared under red light and administered using a red syringe and metal gavage to prevent light exposure. Mice were treated 5 days a week for 9 months beginning at 4 months of age (prior to disease development). As detailed below, a battery of behavioral tests was conducted, including Y-maze spontaneous alternation, open field, rotarod, novel object recognition, Barnes maze, and object location memory.

Pharmacokinetic (PK) Studies

For mouse PK studies, male CD-1 mice (Charles River Canada) were used. Animals were food fasted for 4 h prior to dosing and water fasted 1 h prior to dosing. Mice were dosed by oral gavage under red light at 50 mg/kg JOTROL™ or 50 mg/kg resveratrol (n=12/group). At each time point (15 min, 30 min, 1 h, 4 h) 3 mice of each group were terminally bled via cardiac puncture under red light. Blood was collected into sodium-EDTA tubes under red light and spun at 4.0 for 10 min at 6800×g. Plasma was collected and split into amber tubes. Both left and right brain were collected and snap-frozen in liquid nitrogen and stored at ˜80° C. Drug concentrations were analyzed by liquid chromatography-mass spectrometry (LC-MS/MS) by KDM Laboratories Inc. For rat PK studies, male Sprague-Dawley rats (Charles River Canada) were used. Rats were dosed via oral gavage under red light with either vehicle, 50 mg/kg JOTROL™ or 50 mg/kg MegaResveratrol® (n=3/group). Blood was collected under red light from the trunk at 30 min into K3EDTA tubes and centrifuged immediately at 4° C. for 10 min at 6800×g, and plasma collected into amber tubes. Brains were collected and snap-frozen in liquid nitrogen and stored at ˜80° C. Drug concentration was analyzed by LC-MS/MS.

Behavior

A battery of behavior tests designed to test motor coordination skills, anxiety, spatial learning and reference memory was performed on all long-term-treated mice. Motor coordination was evaluated using rotarod. Anxiety was evaluated by open field and elevated 0-maze tests. Spatial learning and reference memory were tested using Y-maze, novel object recognition (NOR), Object Location Memory (OLM), and Barnes Maze. Video tracking was performed with the automated Etho-Vision XT tracking software (Noldus). Before each behavioral test, mice were habituated to the testing room for 1 h. All arenas were thoroughly cleaned with 70% ethanol between mice. For rotarod test of motor coordination, the Harvard Apparatus Rota Rod was used for this study. This device includes separate lane timers, constant speed and a fixed acceleration rate, mechanical detection of fall, and automatic recording of latencies to fall and rotation speed. The rotarod treadmill was first set at a steady start speed of 1-4 rpm. Each mouse was held by the tail, and placed on the rotating rod, facing away from the direction of the rotation, which allowed the mouse to walk forward and stay upright. When the mouse was touching the rod, it was quickly released, enabling an easy grip. At 3 s after placing the mouse on the rod, acceleration started. Three trials were per-formed with 30 min inter-trial intervals (ITI). Motor coordination was assessed by the latency to fall on the very first trial between treatment groups. Motor learning was measured both within and between sub-jects by comparing the first trial with subsequent trials and is evident as an increased latency to fall over time.

For open field, mice were given 10 min a day for 3 days to explore an arena (40×40 cm). Distance, velocity, and time in center versus periphery were recorded and analyzed. Anxiety-related behavior can be determined by the duration spent in the center of the field versus the periphery. Animals that spend more time in the center than the periphery are considered to have decreased anxiety. For elevated 0-maze, mice are given 5 min to explore an elevated ring-shaped track with walls on two quarters; time spent in open versus closed areas were used to evaluate anxiety-related behavior. Animals who spent more time in the open areas, compared to all time spent on the track, were considered to have less anxious behavior.

Y-maze spontaneous alternation is a classic test of hippocampal function, and measures exploratory behavior based on the willingness of the mice to visit a new arm of the maze rather than a familiar arm. Mice were placed at the center of the Y-shaped maze that contains three solid plastic symmetrical arms (A, B, C) at a 120-degree angle (44 cm length, 8 cm width, 20 cm height) and are given 5 min to explore. Total arm entries and percent of spontaneous alternations were recorded. Arm entry was defined as all four limbs present within the arm. Spontaneous alternation was defined as a set of three arm entries, when each arm entry was to a different arm of the maze, divided by the number of all arm entries, and multiplied by 100. Mice had to make a minimum of 10 arm entries to be considered in the analysis.

NOR tests reference memory. Mice are given 3 min in an open field (40×40 cm) to explore two identical objects. After a 30 min ITI mice were given 5 min in the same open field with one of the same objects (familiar) and a novel object in the place of one of the original objects. Total time spent investigating each object during acquisition and recognition phases was recorded. Time spent exploring the novel object compared to the familiar object (an increase in recognition index) represents increased recognition memory.

OLM tests spatial memory. During habituation trials mice are given 3 min in an open field (40×40 cm) to explore two identical objects. After a 30 min ITI mice were given 5 min in the same open field with one of the objects moved to a different location within the arena. Discrimination index is calculated by the time spent with the object that's in the new location compared to time spent exploring both objects.

Barnes maze tests spatial learning and memory. In brief, mice are placed at the center of a round arena (100 cm in diameter) that contains 20 holes (5 cm diameter) equally spaced around the perimeter (7.5 cm apart), one of which leads to an escape box. The maze is surrounded by visual cues, varying in colors and shapes, and were maintained through-out the course of the experiment. Mice were placed at the center of the table under a box. The timer started once the box was removed, allowing the mouse to explore. Mice were trained during two trials a day for six consecutive days and then tested for memory retention in a probe trial on the 7th day. During the probe trial the escape box was removed, and the table turned 180 degrees. The mouse was given 5 min to explore. The amount of time the mouse spent searching in the escape box location, latency to find the hole, and number of wrong holes visited (errors) were recorded and analyzed. For the two-week retention trial, mice underwent a single trial, 2 weeks after the final probe trial, and time to find the escape box was recorded. The experimenter was blinded to treatment group during behavior and analysis.

Tissue Collection

At study endpoints (age 15 months for sub-chronic study, age 13 months for long-term study), mice were anaesthetized with isoflurane and blood was collected by transcardial puncture. Blood was stored on ice and then centrifuged at 10,000×g at 4° C. for 10 min to collect serum and stored at −80° C. until use. The brain was then extracted and quickly dissected on ice to collect prefrontal cortex (PFC), hippocampus, entorhinal cortex (ERC), and cerebellum, which were immediately frozen in liquid nitrogen. The samples were stored at −80° C. until processed for RNA or protein extraction. For each mouse, brain regions from one hemisphere were used for RNA preparation and qPCR and sections from the other hemisphere were used for protein work including ELISAs.

Protein Extraction

Protein lysates were extracted by sonicating tissue in M-PER Mammalian Protein Extraction Reagent (Thermo Fisher Scientific) supplemented with protease inhibitor (Thermo Fisher Scientific) and phosphatase inhibitor (Thermo Fisher Scientific). Total protein was quantified using Pierce BCA Protein Assay Kit (Thermo Fisher Scientific) and read at 562 nm using EnVision (Perkin Elmer).

ELISA

Human Aβ1-42, Aβ1-40, phosphorylated tau Ser 396, phosphorylated tau Thr181, and total tau were measured in brain tissue by enzyme linked immuno-sorbent assay (ELISA; Invitrogen by Thermo) and normalized to total protein. All the kits were used as per the manufacturer's instructions and measured using the EnVision® multimode plate reader (Perkin Elmer). Serum and brain tissue protein extracts were analyzed for cytokine and chemokine levels using the Immune Monitoring 48-Plex Mouse ProcartaPlex™ Panel (Invitrogen by Thermo) according to manufacturer's standard protocol and read using Luminex xMAP (multi-analyte profiling) technology.

SIRT1 Activity Assay

SIRT1 enzyme activity was measured in extracted protein from the brain using a fluorometric SIRT1 Activity Assay Kit (Abcam) according to manufacturer's protocol. Fluorescence intensity was measured at excitation/emission of 350/460 nm using the EnVision® multimode plate reader (Perkin Elmer). Fluorescence was measured at 5, 10, 20, 30, and 60 min. The greater the fluorescence intensity, the greater the SIRT1 activity in the samples.

Quantitative Real Time PCR (RT-qPCR)

RNA was isolated from tissue using TRIzol reagent (Life Technologies, Thermo Fisher Scientific) and Qiagen RNeasy Mini Kit (Qiagen). RNA concentrations and quality were determined using Nanodrop 2000 UV-Vis Spectrophotometer (Thermo Fisher Scientific). RNA was converted into cDNA using qScript cDNA Synthesis kit (Quantabio). RT-qPCR was run using TaqMan 2-step RT-qPCR reagents (Thermo Fisher Scientific). Samples were amplified for 40 cycles using Quantstudio Flex RT-qPCR system (Applied Biosystems, Thermo Fisher Scientific). Fold-change in gene expression relative to non-transduced controls were normalized. Results presented are based on fold-change using the 2\hat{ }(−ΔΔCt) method. All genes tested by qPCR in this study were amplified with Taqman primer probes (Thermo Fisher Scientific).

Further assessed was RNA from ERC samples using the NanoString Technologies nCounter® Mouse Neuropathology Panel which screens expression of 770 genes specific for neurodegeneration. Assays were performed using the NanoString protocols per the manufacturer's instructions. Data was normalized in nSolver® analysis software to account for systemic variability and further normalized to reference genes to account for sample variability. NanoString nCounter® Advanced Analysis was used to identify differentially expressed genes (DEGs) which were considered significant if they had a p<0.05. Heatmaps were generated using Morpheus Software (Broad Institute).

Statistics

All data are expressed as the mean±SEM. Statistical analyses and graphing were performed with GraphPad Prism 7 (GraphPad Software). Unpaired Student's t test or Mann-Whitney test was used for comparisons of two means. One-way ANOVA with either Dunnett's or Tukey post-hoc analysis was used for multiple comparisons when more than two means were being compared. Repeated measures ANOVA was used to analyze Barnes maze and open field where the same animals were measured over time. Tukey's adjusted P values are presented. Adjusted p <0.05 was deemed to be of statistical significance. For behavioral data, Grubbs' test with an alpha=0.05 was used to test for outliers.

### Results

JOTROL™ displays favorable bioavailability and brain penetration.

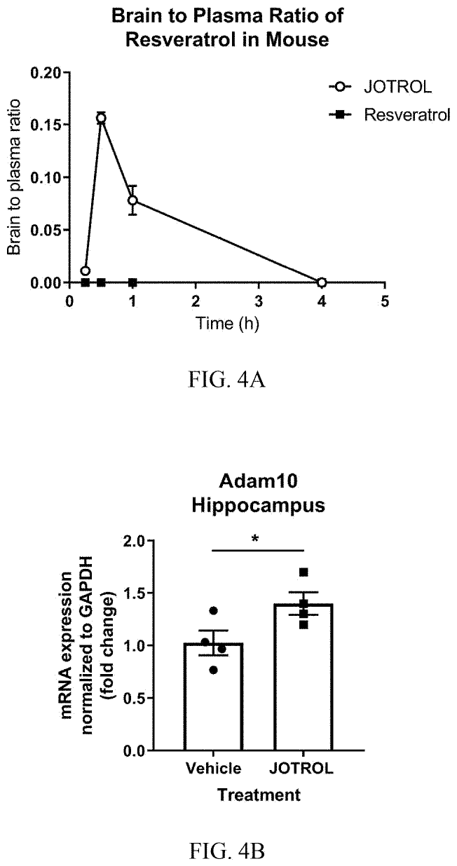

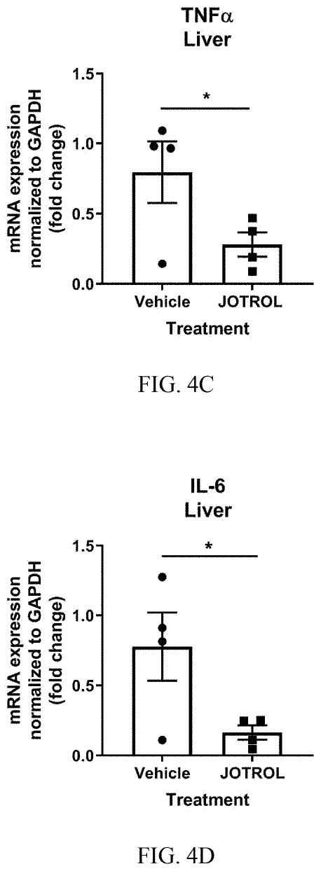

In male wildtype C-1 mice (n=3/time/group), a single dose of 50 mg/kg JOTROL™ showed greater resveratrol accumulation in the brain (p<0.001 at 30 min) when compared to non-formulated resveratrol, which was not detected in the brain (FIG. 4A). Additionally, a single dose of JOTROL™ resulted in greater resveratrol concentration (1417±446.4 ng/mL) in the plasma of Sprague-Dawley rats (n=3/group) 30 min after treatment than non-formulated resveratrol (179.7±33.63 ng/mL, p<0.05).

Sub-chronic treatment with JOTROL™ improves AD-related genes in 15-month-old male 3×Tg-AD mice.

In order to investigate the short-term effects of JOTROL™ treatment in aged male 3×Tg-AD mice. 15-month-old mice (n=5/group) were treated orally with 50 mg/kg JOTROL™ or vehicle for 36 days. Brain, liver, and spleen were analyzed for AD pathology and inflammation using qPCR (FIG. 4A-4D). In the hippocampus of 3×Tg-AD mice, gene expression of the α-secretase, Adam10, was increased in JOTRO™L™-treated mice (p<0.05). Additionally, expression of pro-inflammatory cytokines Tnfa and 116 were decreased in the livers of JOTROL™ mice (p<0.05 for both). Furthermore, expression of Tnfa and 116 were significantly decreased in the spleen of JOTROL™-treated mice.

Additionally, acute (30 min) JOTROL™ treatment improved the expression of AD-related genes in the brains of male Sprague-Dawley rats (n=3/group) compared to resveratrol and vehicle. The anti-amyloidogenic gene Adam10 and Sirt1 expression were both increased in rat brains while tau gene expression was decreased.

Prophylactic Effects of JOTROL™ on AD Prevention.

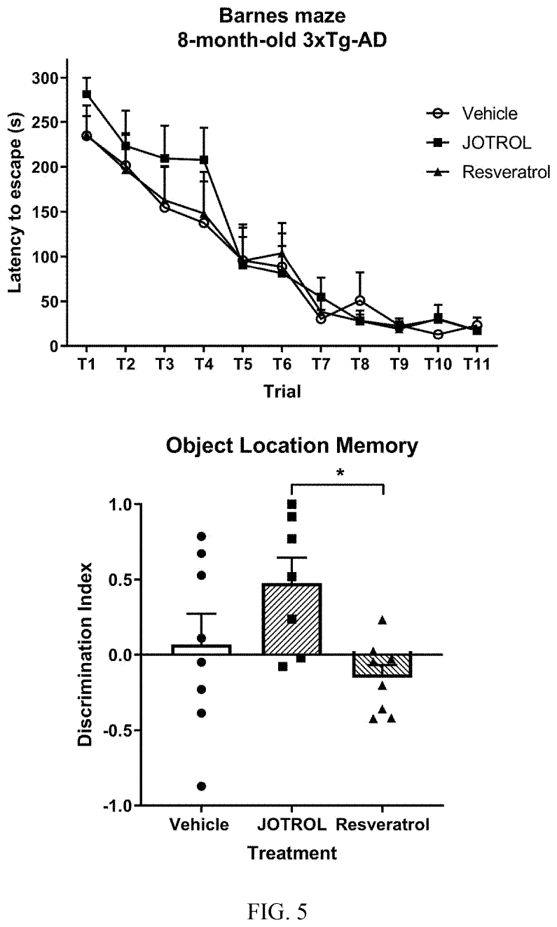

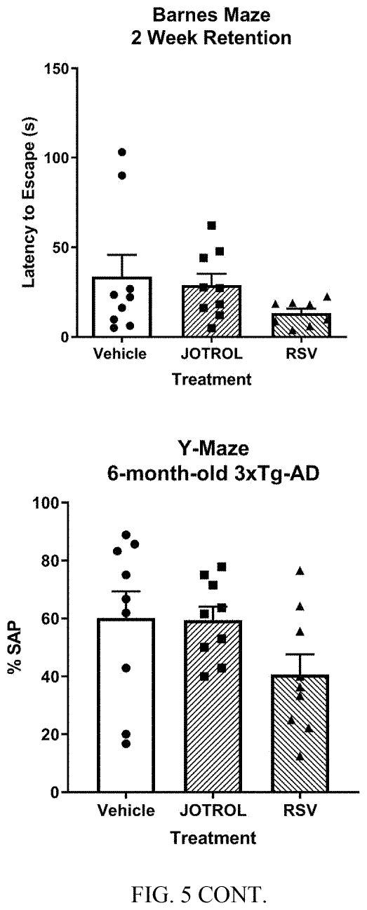

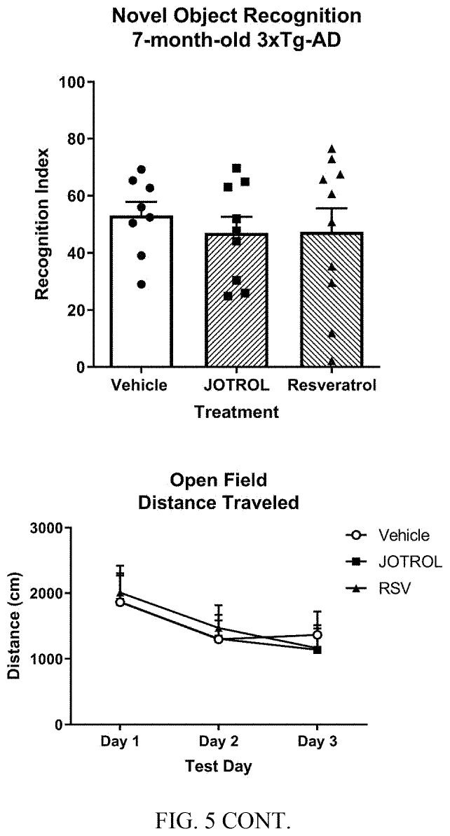

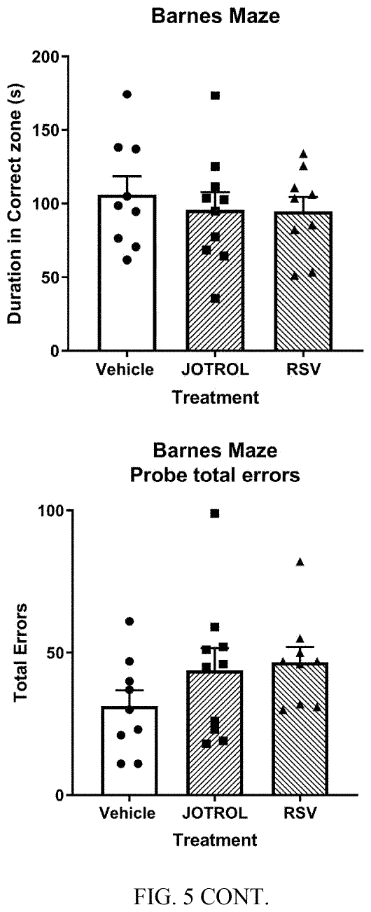

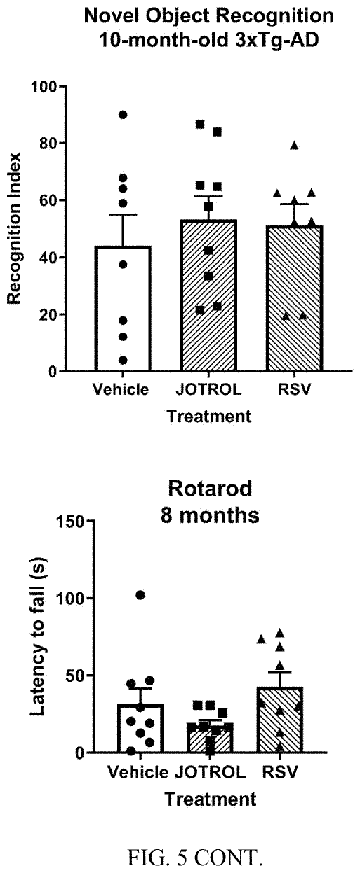

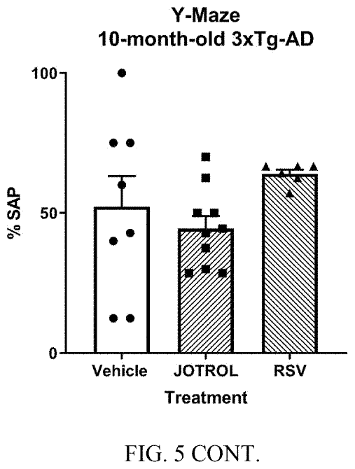

In order to test whether prophylactic treatment with JOTROL™ can prevent AD-related cognitive deficits in 3×Tg-AD mice, such as in learning and memory (FIG. 5). Four-month-old (prior to development of disease) male and female 3×Tg-AD mice were treated daily with JOTROL™, RSV, or vehicle for 9 months. Memory and learning-related behavior was evaluated using Y-maze, Barnes maze, sub-threshold NOR, and OLM. A significant improvement in the OLM discrimination index, a measure of spatial memory, was observed in mice receiving JOTROL™ treatment compared to RSV treated mice (0.63±0.2, p<0.05) at 10 months of age. This significant relationship was maintained in females when analyzed separately from males (p<0.05). No significant group differences were observed in Y-maze spontaneous alternation performance at 6 or 10 months of age.

Generally, classic NOR tests hippocampal-dependent learning; sub-threshold NOR, as used herein, measures learning and memory under conditions that are normally insufficient for encoding and memory formation. Since the 3×Tg-AD mice were not showing significant impairment of learning and memory capabilities, which has been reported in this mouse model, if JOTROL™ treatment could enhance learning and memory behavior in these mice, past subthreshold, was investigated. Sub-threshold NOR was performed at 7 and 10 months of age [F(2,24)=0.238, p>0.05 at 7 months; F(2,22)=0.29, p>0.05 at 10 months]. When results were analyzed separately by sex there was a significant difference in NOR discrimination index between JOTROL™ and vehicle-treated mice (p<0.01). JOTROL™-treated mice showed an improvement while vehicle-treated mice favored the familiar object to an extent that may represent the vehicle mice group tendency for neophobia fear of new objects. Barnes maze was performed at 8 months of age and no differences were seen between performance as measured by duration spent in the escape zone [F(2,25)=0.304, p=0.74], total errors made [F(2,25)=1.572, p >0.05], and latency to escape measured 2 weeks after the probe trial [F(2,25)=0.563, p>0.05] measured by one-way ANOVA followed by Tukey post-hoc.

Treatment with either compound resulted in no adverse effects on sensorimotor behavior, evaluated by rotarod and open field (FIG. 5), which were unchanged between treatment groups or weight loss. Over the course of treatment, four mice were prematurely euthanized (two males in the vehicle group and two males in the RSV group). Two mice due to splenomegaly, which is common in the 3×Tg-AD mouse, one due to skin wounds acquired from aggressive behavior, and one mouse due to intestinal blockage (male RSV group). Considering that mice were euthanized in both vehicle and RSV treated groups, it is unlikely that the conditions were related to treatment.

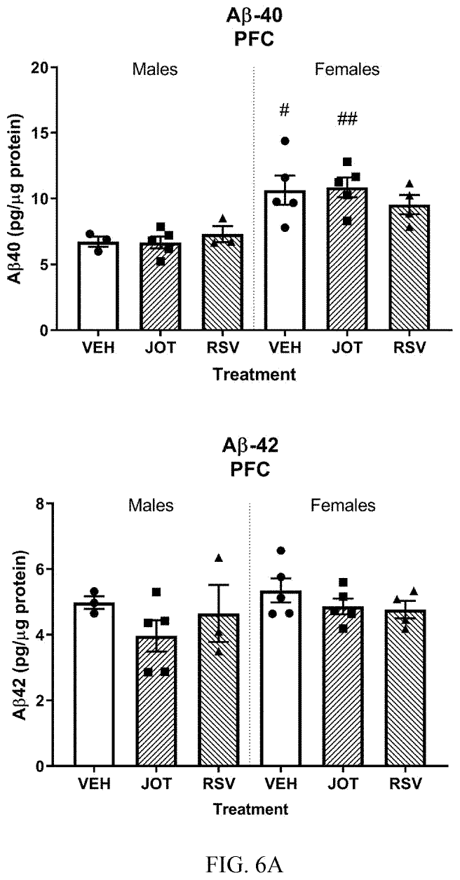

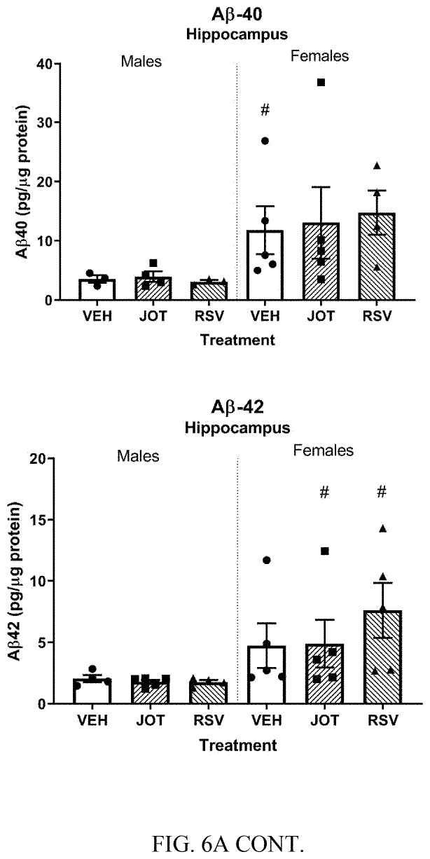

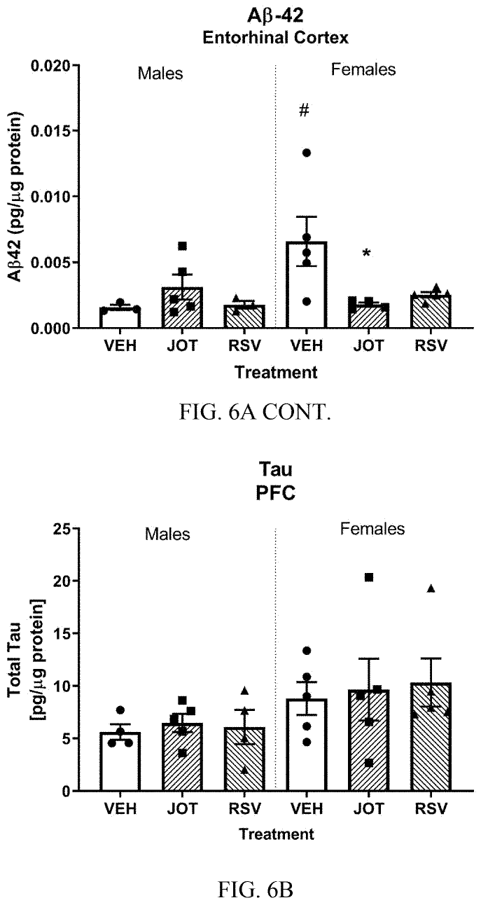

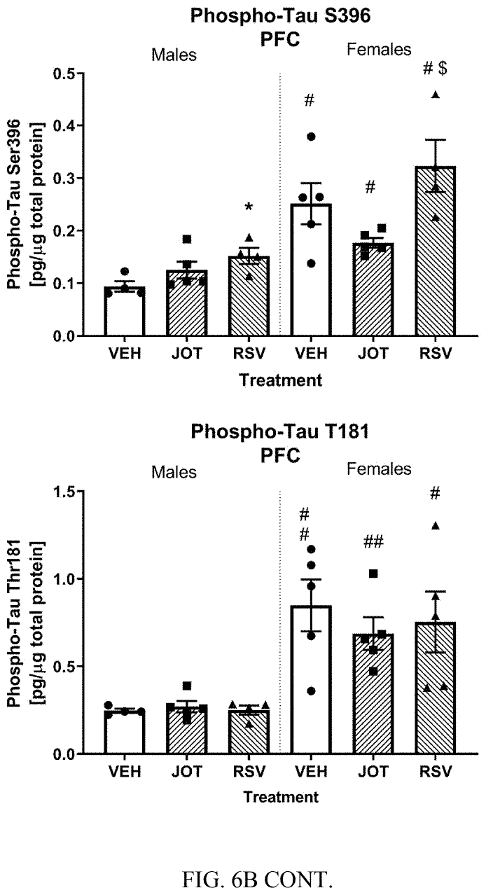

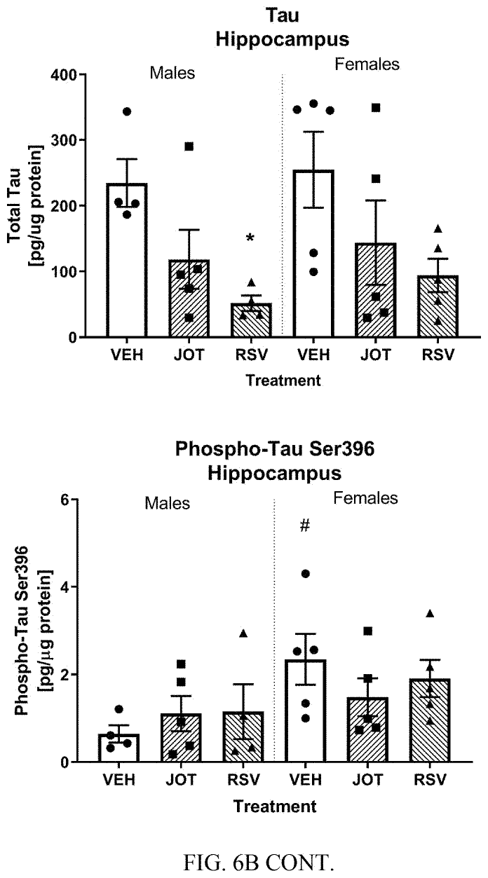

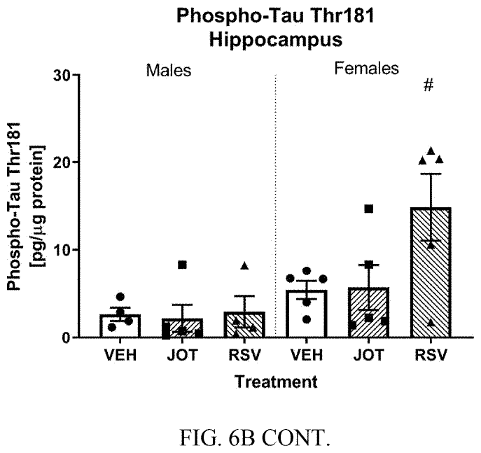

Next, whether long-term treatment with JOTROL™ improved AD-related pathology in male and female 3×Tg-AD mice was investigated. Af3 and hyperphosphorylated tau are the two most common pathological hallmarks associated with AD. Soluble Af342 was significantly decreased in the ERC of JOTROL™-treated females (p<0.05) compared to vehicle females, although this pattern was not seen in the males (FIG. 6A, 6B). Levels of soluble Af340 and Af342 in the PFC and hippocampus were not significantly changed in the treated animals (FIG. 6A, 6B). Total tau was decreased in the hippocampus of RSV-treated males (182.8±53.66, p<0.05; FIG. 6A, 6B), and was significantly decreased in JOTROL™ (114.6±43.7, p<0.05) and RSV (170.5±44.83, p<0.01) treated mice compared to vehicle when males and females were analyzed together. Phosphorylated tau at Ser396 was significantly decreased in the PFC of JOTROL™ compared to RSV-treated female mice (0.146±0.05, p<0.05; FIG. 6A, 6B).

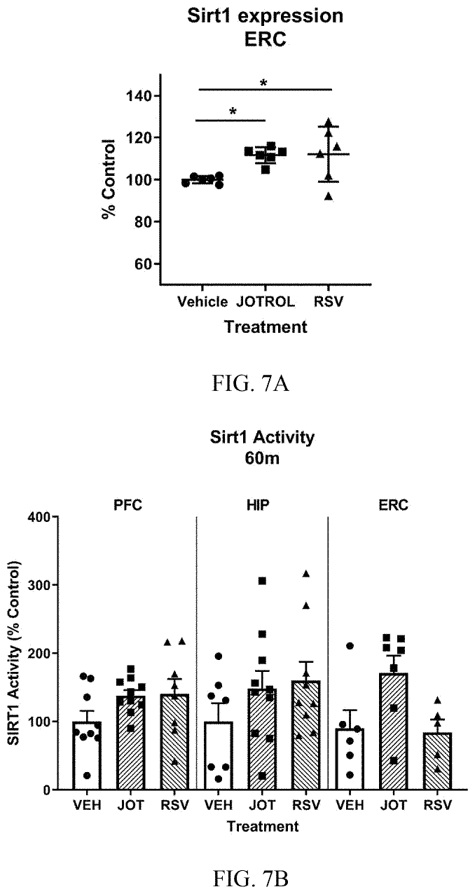

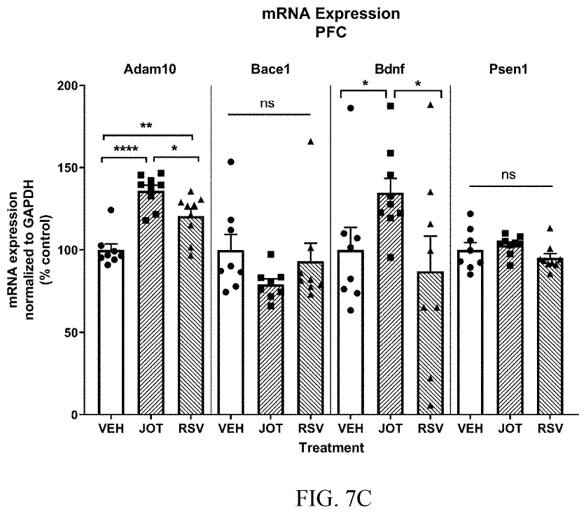

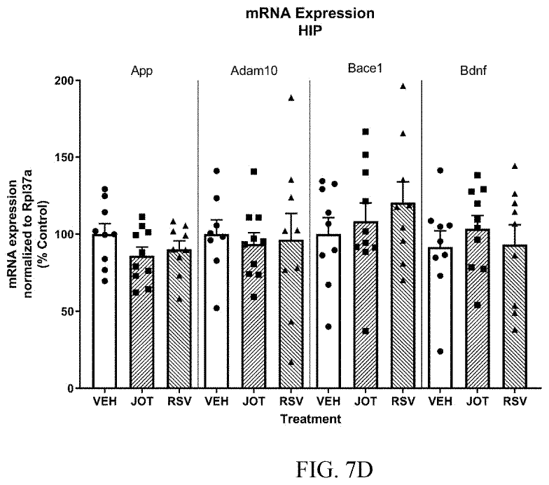

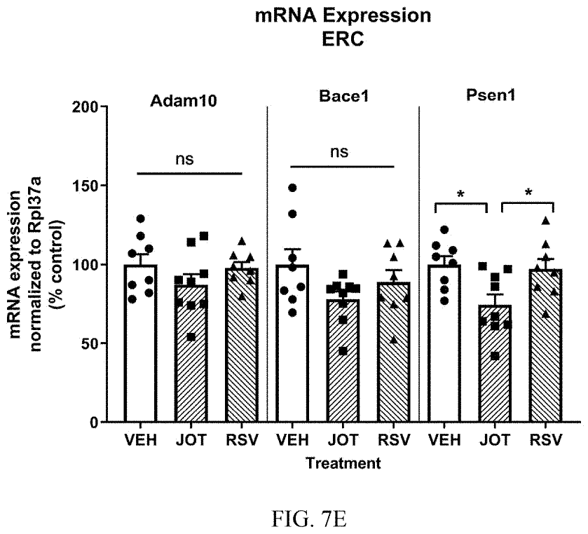

JOTROL™ treatment improves AD-related gene expression. 13-month-old male and female 3×Tg-AD mice treated for 9 months with daily JOTROL™ (50 mg/kg) showed a trend for increased SIRT1 activity in PFC (37.84%±21.11%, p >0.05), hippocampus (48.27%±38.93%, p>0.05), and ERC (81.1%±33.67%, p>0.05) compared to vehicle-treated control animals (FIG. 7B). Sirt1 gene expression was increased in the ERC of both JOTROL™ (11.59%±4.552%, p<0.05) and RSV (12.07%±4.552%, p<0.05) treated mice compared to vehicle (FIG. 7A). AD-related genes showed sex-specific and brain-region dependent changes in expression (FIG. 7C-7E). In the PFC, one of the first brain regions to accumulate Aβf3 species in the 3×Tg-AD mouse, Adam10, a gene involved in non-amyloidogenic AβPP processing, is significantly increased in JOTROL™ (35.96%±5.548%, p<0.0001) and RSV (20.44%±5.548%, p<0.01) treated mice compared to vehicle. This increase in expression is greater in the treated female mice (p<0.0001) when analyzed separately by sex. Bdnf expression is significantly increased in the PFC of JOTROL™-treated (34.63%±15.87%, p<0.05) mice compared to vehicle and RSV-treated mice. In male PFC, Bace1, a gene involved in amyloidogenic Af3PP processing, is significantly decreased (p<0.05) in JOTROL™-treated mice. In the ERC, Psen1, a gene involved in amyloidogenic Af3PP processing, is significantly decreased in JOTROL™-treated mice compared to vehicle (25.56%±8.737%, p<0.05) and RSV-treated (22.68%±8.737%, p<0.05) mice. Mean expression of Adam10 and Bace1 was unchanged between treatment groups [F (2,22)=1.413, p>0.05; F (2,22)=2.187, p >0.05, respectively]. In the hippocampus, expression of genes involved in AβPP processing, Adam10 [F(2,25)=0.077, p>0.05] and Bace1 [F(2,25)=0.713, p>0.05], were unchanged between treatment groups, as was App [F(2,25)=1.05, p>0.05] and Bdnf [F(2,24)=0.367, p>0.05] expression.

JOTROL™ treatment causes changes in inflammatory markers in the brain and periphery.

AD is accompanied by central and peripheral inflammation and resveratrol has previously been shown to decrease pro-inflammatory cytokines. In the present study, serum and entorhinal cortex was evaluated for 48 cytokines and chemokines using multiplex ELISAs. Table 3 displays changes in serum cytokine protein levels in response to treatment, separated by sex due to sex differences in cytokine levels. Male mice treated with JOTROL™ showed significant decreases in IL-9 (−12.94±4.95 pg/mL, p<0.05), LIX (−6939±2230 pg/mL, p<0.05), MCP-3 (−97.26±33.47 pg/mL, p<0.05), and increased MIP-1a (12.1±4.44 pg/mL, p<0.05) compared with vehicle-treated males. Female mice treated with JOTROL™ displayed a significant decrease in serum levels of IL-10 (−15.53±5.73 pg/mL, p<0.05), LIF (−4.31±1.64 pg/mL, p<0.05), and MCP-3 (−327.1±140.7 pg/mL, p<0.05) compared to vehicle-treated females. RSV-treated males showed significant changes in IL-22 (−23.37±5.2 pg/mL, p<0.05), IL-3 (−1.53±0.26 pg/mL p<0.01), MCP-3 (−120.8±39.78 pg/mL, p<0.05), and MIP-2 (−16.16±5.95 pg/mL, p<0.05) compared to vehicle males.

TABLE 3
Changes in serum cytokine levels (pg/mL) with treatment compared to vehicle, separated
by sex; n = 3-5/group; p-value calculated by unpaired t-test, significant values in bold

| Analyte | JOT M | p | RSV M | p | JOT F | p | RSV F | p |
|---|---|---|---|---|---|---|---|---|
| BAFF | −109.80 | 0.725 | −44.06 | 0.910 | −142.80 | 0.214 | 22.01 | 0.882 |
| Eotaxin | 396.40 | 0.643 | 943.10 | 0.248 | −3170.00 | 0.128 | −3162.00 | 0.026* |
| G-CSF | 7.35 | 0.221 | −5.14 | 0.244 | 2.07 | 0.373 | −0.68 | 0.634 |
| IFNg | 1.84 | 0.258 | −0.66 | 0.540 | 1.99 | 0.330 | 1.06 | 0.280 |
| IL-10 | 7.02 | 0.795 | 2.25 | 0.935 | −15.53 | 0.030* | −8.54 | 0.289 |
| IL-12p70 | −8.65 | 0.253 | −13.72 | 0.174 | 0.14 | 0.945 | 0.15 | 0.951 |
| IL-13 | −66.50 | 0.088 | −72.28 | 0.169 | −3.67 | 0.562 | 15.44 | 0.314 |
| IL-15/IL-15R | −8.99 | 0.140 | −11.93 | 0.081 | 8.98 | 0.111 | 0.51 | 0.892 |
| IL-17A | −1.41 | 0.635 | −6.64 | 0.073 | −1.11 | 0.749 | −5.14 | 0.115 |
| IL-18 | −418.30 | 0.367 | −415.10 | 0.453 | −220.80 | 0.246 | −74.10 | 0.741 |
| IL-19 | −5.17 | 0.901 | −23.06 | 0.672 | 4.27 | 0.807 | −17.31 | 0.262 |
| IL-1b | −2.32 | 0.325 | −5.86 | 0.235 | 11.04 | 0.240 | 0.80 | 0.737 |
| IL-22 | 13.96 | 0.476 | −23.37 | 0.011 | 3.59 | 0.841 | −2.84 | 0.804 |
| IL-23 | 49.02 | 0.362 | −12.74 | 0.324 | 4.04 | 0.615 | 16.41 | 0.220 |
| IL-25/IL-17 | −86.44 | 0.478 | −235.80 | 0.132 | 32.28 | 0.526 | −12.62 | 0.705 |
| IL-27 | 5.98 | 0.689 | −12.09 | 0.306 | −2.87 | 0.240 | 0.92 | 0.873 |
| IL-28 | 6.72 | 0.892 | −44.41 | 0.325 | 27.67 | 0.677 | 5.70 | 0.890 |
| IL-2RA | −15.43 | 0.178 | −21.16 | 0.092 | −38.83 | 0.396 | −71.13 | 0.054 |
| IL-3 | 0.24 | 0.867 | −1.53 | 0.002** | 0.52 | 0.199 | 0.15 | 0.583 |
| IL-33 | −79.35 | 0.796 | 107.60 | 0.840 | 60.44 | 0.553 | −4.36 | 0.975 |
| IL-5 | −2.93 | 0.795 | 8.72 | 0.725 | −2.67 | 0.950 | 0.05 | 0.999 |
| IL-6 | −67.00 | 0.146 | −60.32 | 0.260 | −4.31 | 0.739 | −10.05 | 0.445 |
| IL-9 | −12.94 | 0.047* | −9.66 | 0.178 | −3.09 | 0.531 | 13.83 | 0.430 |
| IP-10 | −53.25 | 0.134 | −47.95 | 0.378 | −15.59 | 0.598 | −9.75 | 0.735 |
| KC | −62.91 | 0.520 | −90.17 | 0.406 | −12.46 | 0.834 | −41.09 | 0.247 |
| LIF | −7.97 | 0.088 | −8.06 | 0.077 | −4.31 | 0.030* | −1.86 | 0.414 |
| LIX | −6939.00 | 0.027* | −6715.00 | 0.064 | −1475.00 | 0.555 | 1701.00 | 0.647 |
| Leptin | 14549.0 | 0.311 | −1768.00 | 0.739 | −15376.0 | 0.474 | −33707.0 | 0.006** |
| MCP-1 | 70.56 | 0.196 | −28.90 | 0.501 | −48.27 | 0.138 | −43.64 | 0.177 |
| MCP-3 | −97.26 | 0.034* | −120.80 | 0.039* | −327.10 | 0.049* | −212.20 | 0.126 |
| MIP-1a | 12.10 | 0.034* | 4.01 | 0.399 | 1.46 | 0.395 | 3.71 | 0.167 |
| MIP-1b | 19.27 | 0.111 | −6.40 | 0.388 | 2.55 | 0.405 | 0.17 | 0.862 |
| MIP-2 | −7.45 | 0.196 | −16.16 | 0.035* | −16.50 | 0.120 | −7.86 | 0.212 |
| RANTES | −23.65 | 0.140 | −24.75 | 0.265 | −46.38 | 0.133 | −9.09 | 0.789 |
| TNFa | 7.19 | 0.131 | −0.08 | 0.987 | −6.37 | 0.126 | −2.67 | 0.589 |
| VEGF-A | −17.81 | 0.121 | −17.68 | 0.140 | −6.06 | 0.477 | 3.32 | 0.832 |
| sRANKL | −20.08 | 0.132 | 4.06 | 0.828 | −3.18 | 0.810 | −0.58 | 0.968 |

RSV-treated females displayed significant changes in Eotaxin (−3162±1165 pg/mL, p<0.05) and Leptin (−33707±9226 pg/mL, p<0.01) compared to vehicle females. Serum cytokine levels were detected. Changes in cytokine levels in the entorhinal cortex are displayed in Table 4. No significant changes in cytokine expression, were observed in JOTROL™-treated males. JOTROL™-treated females had significantly increased levels of IL-23 (37.76±11.72 pg/mL, p<0.05), IL-28 (96.86±37.22 pg/mL, p<0.05), and VEGF-A (2.78±0.71 pg/mL, p<0.05) compared to vehicle females. RSV-treated males showed significantly increased levels of IL-2RA (0.77±0.043 pg/mL, p<0.001) and MIP-2 (7.51±2.55 pg/mL, p<0.05). RSV-treated females displayed highly significant changes in ERC cytokine levels, with an increase a decrease in IL-18 (−84.91±35.55 pg/mL, p<0.05), and increased levels of IL-19 (105.7±22.58 pg/mL, p<0.01), IL-23 (42.53±10.42 pg/mL, p<0.01), IL-28 (146.9±34.42 pg/mL, p<0.01), IL-6 (5.44±2.1 pg/mL, p<0.05), IL-7 (40.12±15.04 pg/mL, p<0.05), and IP-10 (21.13±4.32 pg/mL, p<0.01) compared to vehicle females.

TABLE 4
Changes in ERC cytokine levels (pg/mL) with treatment compared
to vehicle, separated by sex; n = 3-5/group; p-value
calculated by unpaired t-test, significant values in bold

| Analyte | JOT M | p | RSV M | p | JOT F | p | RSV F | p |
|---|---|---|---|---|---|---|---|---|
| IFNg | −0.10 | 0.884 | 1.51 | 0.169 | −1.05 | 0.290 | −0.64 | 0.495 |
| IL- 10 | 5.60 | 0.448 | 33.20 | 0.098 | −6.70 | 0.570 | 0.54 | 0.966 |
| IL-12p70 | 0.32 | 0.588 | 0.61 | 0.352 | 0.47 | 0.597 | 1.75 | 0.123 |
| IL-13 | 0.01 | 0.997 | 5.16 | 0.216 | −1.51 | 0.702 | 1.74 | 0.640 |
| IL-17A | 0.60 | 0.405 | 2.77 | 0.177 | −0.56 | 0.739 | 0.69 | 0.702 |
| IL-18 | 7.38 | 0.816 | 42.33 | 0.229 | −34.49 | 0.477 | −84.91 | 0.048 |
| IL-19 | 35.73 | 0.440 | 104.30 | 0.213 | 66.80 | 0.086 | 105.70 | 0.003** |
| IL-1b | 0.80 | 0.566 | 0.97 | 0.075 | −0.88 | 0.139 | −1.12 | 0.061 |
| IL-2 | 8.62 | 0.432 | 31.55 | 0.152 | 0.13 | 0.987 | 18.07 | 0.104 |
| IL-22 | 2.19 | 0.402 | 2.18 | 0.367 | −1.90 | 0.180 | −0.13 | 0.953 |
| IL-23 | 8.91 | 0.614 | 41.51 | 0.275 | 37.76 | 0.018* | 42.53 | 0.006** |
| IL-25/IL-17 | 42.20 | 0.191 | 15.36 | 0.116 | −33.38 | 0.076 | −23.30 | 0.216 |
| IL-27 | 2.60 | 0.074 | 2.75 | 0.114 | −0.31 | 0.744 | −0.87 | 0.194 |
| IL-28 | 62.16 | 0.193 | 130.90 | 0.242 | 96.84 | 0.041* | 146.90 | 0.004** |
| IL-2RA | 0.58 | 0.054 | 0.77 | 0.0001*** | −0.52 | 0.329 | −0.95 | 0.135 |
| IL-3 | 0.28 | 0.532 | 0.18 | 0.370 | −0.29 | 0.140 | −0.34 | 0.102 |
| IL-33 | 43.04 | 0.212 | 64.64 | 0.065 | −39.53 | 0.129 | −56.09 | 0.056 |
| IL-5 | 2.80 | 0.408 | 3.37 | 0.264 | −6.15 | 0.110 | −6.60 | 0.058 |
| IL-6 | 2.09 | 0.602 | 2.70 | 0.547 | 4.51 | 0.103 | 5.44 | 0.036* |
| IL-7 | 0.16 | 0.993 | 41.08 | 0.231 | 25.92 | 0.203 | 40.12 | 0.045* |
| IL-9 | 5.93 | 0.364 | 19.29 | 0.149 | 8.23 | 0.327 | 7.25 | 0.402 |
| IP-10 | 1.70 | 0.848 | 13.80 | 0.308 | 13.75 | 0.056 | 21.13 | 0.002** |
| LIX | 3.65 | 0.588 | 16.39 | 0.073 | −7.63 | 0.258 | −12.67 | 0.072 |
| M-CSF | −0.14 | 0.607 | 0.50 | 0.220 | −0.27 | 0.648 | −0.32 | 0.571 |
| MCP-3 | 0.01 | 0.991 | 0.76 | 0.612 | 4.36 | 0.297 | 1.94 | 0.064 |
| MIP-2 | 3.58 | 0.316 | 7.51 | 0.042* | −3.39 | 0.297 | −2.59 | 0.448 |
| RANTES | −1.28 | 0.675 | 1.40 | 0.606 | −0.93 | 0.657 | −1.97 | 0.375 |
| TNFa | 7.70 | 0.218 | 23.73 | 0.057 | −11.83 | 0.099 | −10.12 | 0.170 |
| VEGF-A | 0.42 | 0.811 | 3.55 | 0.210 | 2.78 | 0.011* | 4.80 | 0.074 |
| sRANKL | −0.79 | 0.464 | 2.59 | 0.249 | −0.29 | 0.868 | 0.70 | 0.682 |

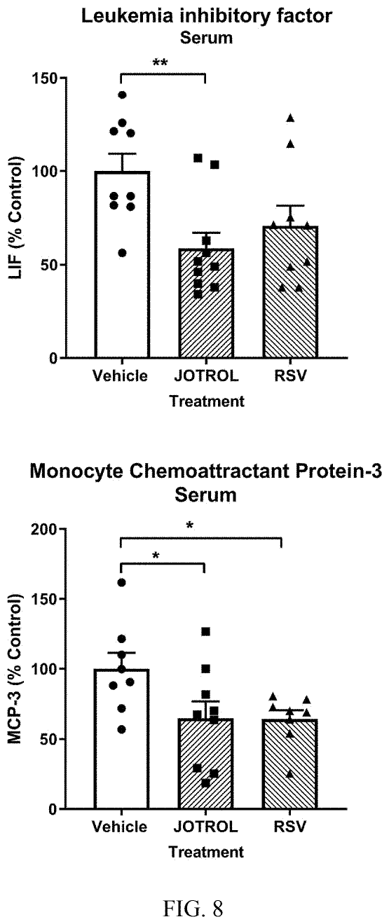

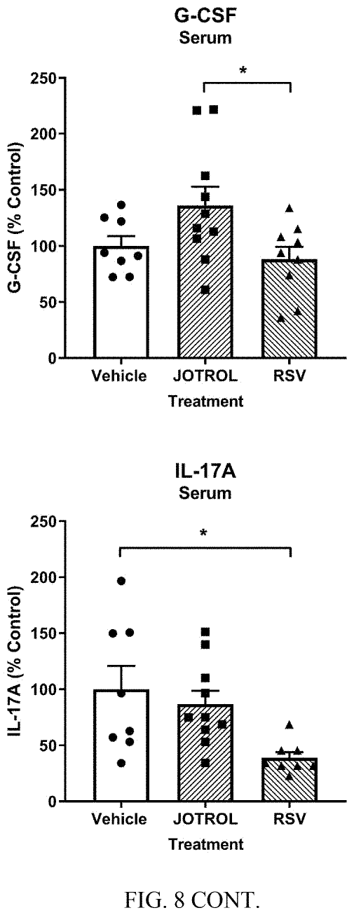

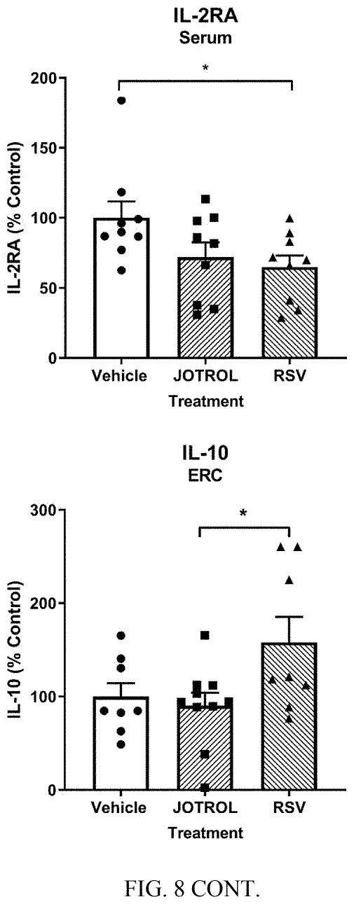

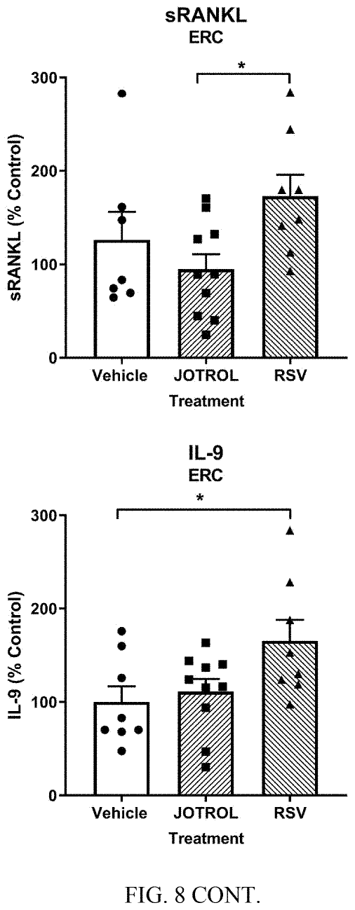

Significant group differences are graphed in FIG. 8. When males and females are combined using percent control, Leukemia Inhibitory Factor (LIF), a member of the IL-6 family cytokines, is significantly decreased in the serum of JOTROL™ mice compared to vehicle (−41.2±13.15 pg/mL, p<0.01). Monocyte chemoattractant protein-3 (MCP-3; aka CCL7), and inflammatory cytokine, is decreased in JOTROL™ serum versus vehicle mice (−35.28±14.58 pg/mL, p<0.05). Granulocytes colony-stimulating factor, which in the CNS can promote neurogenesis, increase neuroplasticity and inhibit apoptosis, was significantly increased in the serum of JOTROL™ versus RSV-treated mice (47.77±18.22 pg/mL, p<0.05). The pro-inflammatory cytokine, interleukin-17A (IL-17A) was decreased in RSV-treated mice compared to vehicle (61.02±20.37 pg/mL, p<0.05). IL-2 receptor subunit alpha is also significantly decreased in serum of RSV-treated mice compared to vehicle (35.08±14.42 pg/mL, p<0.05).

In the ERC, there were significant differences between JOTROL™ and RSV-treated mice in IL-10 (67.86±26.33 pg/mL, p<0.05) and soluble receptor activator of nuclear factor-KB ligand levels (78.18±30.42 pg/mL, p<0.05). RSV-treated mice also had increased levels of IL-9 compared to vehicle (65.4±25.69 pg/mL, p<0.05).

JOTROL™ treatment results in gene expression changes.

Heatmaps generated from this experiment illustrate the 41 differentially expressed genes (DEGs) in the ERC that were significant between JOTROL™ and vehicle-treated mice, and the 71 DEGs that were significant between RSV and vehicle-treated mice. The top 15 DEGs for each treatment group were graphed by fold-change com-pared to vehicle. Genes that were significantly changed between JOTROL™ and vehicle mice include those involved in protein metabolism (Cast, Gsn, Gusb, Sgpl1), transcriptional regulation (Acin1, Hdac1, Hdac8, Taf4, Nfe212, Npas4, Xbp1), and copper homeostasis (Atp7a, Cp).

When analyzed separately by sex, male JOTROL™-treated mice displayed 61 significant DEGs and female JOTROL™-treated mice displayed 18 significant DEGs compared to vehicle. Male JOTROL™ significant DEGs included genes involved in inflammatory response (C1qa, C1qc, Atm, Csf1r), genes involved in neurotransmitter signaling (Cacna1a, Chat, Cntf, Drd2, Gria3, Brik2, Itpr2), and genes involved in transcriptional regulation (Ep300, Erg, Hdac1, Mecp2, Nfe212, Npas4, Sf3b4, Sirt1, Sirt2, Tada2b, Tbpl1). Female JOTROL™ DEGs consist of genes related to transcriptional regulation, including cAMP-response element binding protein (CREB) regulated transcription coactivator 2 (Crtc2) which promotes transcription of CREB-targeted genes, CREB binding protein (Crebbp) which encodes a protein that acetylates histone and non-histone proteins, the transcription factor Sp3, and the RNA polymerase Polr2h.

To summarize, JOTROL™ was created to overcome the low bioavailability of non-formulated resveratrol. In the present study, superior oral bioavailability of JOTROL™ over non-formulated trans-resveratrol was shown in two rodent models. It was observed that both sub-chronic and long-term treatment with JOTROL™ improves several AD-related genes and impacts central and systemic inflammation in the 3×Tg-AD mouse model. These findings show sex-specific and brain region-dependent changes in AD-related pathways as a result of long-term JOTROL™ treatment. Accumulation of hyperphosphorylated tau is a pathological characteristic of AD, and in the present study, a significant decrease in total tau in the hippocampus of JOTROL™-treated mice compared to vehicle was observed. In the present study, it was shown that JOTROL™, but not RSV, significantly increases the expression of Bdnf, a molecule which has been shown to positively impact memory and cognitive function. In contrast, BDNF has not been shown to increase with resveratrol treatment in other studies. The results of the present study also showed that short-term (36 days) JOTROL treatment was enough to lower expression of pro-inflammatory cytokines Tnf and Il6 in the liver of aged 3×Tg-AD mice.

### OTHER EMBODIMENTS

Any improvement may be made in part or all of the method steps, compositions, and formulations. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A method of enhancing the bioavailability of resveratrol in a subject, comprising orally administering to the human subject a resveratrol solubulization product formulation consisting of: resveratrol; an emulsifying agent mixture of polysorbate 80 and polysorbate 20; at least one medium-chain triglyceride (MCT); and tocopherol or mixed tocopherols, wherein the formulation is orally administered to the human subject under fasting conditions.

2. The method of claim 1, wherein the resveratrol administered is in an amount from about 200 mg to about 700 mg per dose.

3. The method of claim 2, wherein the resveratrol is in an amount of about 500 mg per dose.

4. The method of claim 1, wherein the formulation is administered at least once daily.

5. The method of claim 1, wherein the formulation is formulated as a soft gelatin capsule, a hard gelatin capsule, a soft gelatin-free capsule, or a hard gelatin-free capsule.

6. The method of claim 1, wherein the resveratrol exhibits an AUC_{O-t} which is about 500 h*ng/ml to no more than about 2100 h*ng/ml following administration of the formulation to the human subject under fasting conditions, wherein t is between about 1 and about 24 hours.

7. The method of claim 1, wherein the resveratrol exhibits a Cmax which is about 220 ng/ml to about 400 ng/ml following administration of the formulation to the human subject under fasting conditions.

8. The method of claim 1, wherein the resveratrol exhibits an AUC_{(0-infin.)} which is about 500 h*ng/mL to no more than about 2100 h*ng/mL following administration of the formulation to the human subject under fasting conditions.

9. The method of claim 1, wherein the fasting conditions comprise fasting the subject for at least 2 hours immediately prior to administering the formulation, and for at least another 1 hour immediately after administering the formulation.

10. An oral pharmaceutical composition comprising at least one dose of a resveratrol solubulization product formulation consisting of: about 200 to about 700 mg of resveratrol; an emulsifying agent mixture of polysorbate 80 and polysorbate 20; at least one medium-chain triglyceride (MCT); and tocopherol or mixed tocopherols, wherein the formulation is formulated as a soft gelatin capsule, a hard gelatin capsule, a soft gelatin-free capsule, or a hard gelatin-free capsule, and wherein said composition upon oral administration to a human subject under fasting conditions, provides at least one of the following pharmacokinetic parameters: a. AUC₍₀₋ₜ₎ of at least about 500 h*ng/mL; b. AUC_{(0-infin.)} of no more than about 2100 h*ng/mL; and c. Cmax of at least about 220 ng/ml, wherein t is between about 1 and about 24 hours.

11. The oral pharmaceutical composition of claim 10, wherein the composition comprises multiple doses of the resveratrol solubulization product formulation.

12. A method for the treatment of at least one neuroinflammatory disorder, comprising orally administering to a human subject in need thereof the oral pharmaceutical composition of claim 10 or claim 11, wherein the formulation is orally administered to the human subject under fasting conditions.

13. The method of claim 12, wherein the at least one neuroinflammatory disorder is selected from the group consisting of: Ataxia, Alzheimer's disease, Mild Cognitive Impairment, ALS, Parkinsonism, an acute neurologic injury, Traumatic Brain Injury including concussion, a Lysosomal Storage Disease, mucopolysaccharidosis, a mitochondrial function disorder, MELAS, LHON, hearing loss, and speech acuity.

14. A method of enhancing the bioavailability of resveratrol in a subject, comprising orally administering to the human subject a resveratrol solubulization product formulation under fasting conditions.
